(19) United States
(12) Patent Application Publication
(10) Pub. No.: US 2022/0047525 A1
(43) Pub. Date: Feb. 17, 2022

(54) TRANSDERMAL PHARMACEUTICAL FORMULATIONS OF NABILONE ALONE AND IN COMBINATION WITH CANNABINOIDS

(71) Applicant: Pike Therapeutics, Inc., Vancouver (CA)

(72) Inventors: Fotios M. Plakogiannis, Whitestone, NY (US); Nisarg Modi, Jersey City, NJ (US)

(21) Appl. No.: 17/401,598

(22) Filed: Aug. 13, 2021

Related U.S. Application Data
(60) Provisional application No. 63/066,386, filed on Aug. 17, 2020.
(60) Provisional application No. 63/229,021, filed on Aug. 3, 2021.

(51) Int. Cl. A61K 9/70; A61K 31/352; A61K 31/05; A61K 9/00

(52) U.S. Cl. CPC A61K 9/7069; A61K 9/0021; A61K 31/05; A61K 31/352

(57) ABSTRACT

The present disclosure relates to the to the transdermal administration of nabilone and cannabinoids and derivatives of these compounds, for the treatment and/or prevention and/or control of medical conditions.

This application claims benefit of U.S. Ser. No. 63/066,386, filed Aug. 17, 2020, and U.S. Ser. No. 63/229,021, filed Aug. 3, 2021, the entireties of which are incorporated herein by reference.

### BACKGROUND OF THE INVENTION

The present disclosure relates to the transdermal administration of, for example nabilone and cannabinoids and derivatives of these compounds, for the treatment and/or prevention and/or control of medical conditions. Cannabis (marijuana) is a schedule-I drug in USA. Cannabis is a flowering plant which contains more than 400 phytonutrient (micronutrient). More than 100 different types of terpenoids, essential oils, antioxidants and cannabinoids have been extracted from the plant. From all of the phytochemicals, only tetrahydrocannabinol (THC) showed significant psychoactive effect. A number of research papers have been published on THC due to its psychoactive and therapeutic effects. Apart from THC, several other constituents have been studied, which also showed some therapeutic effect without psychoactive effect such as cannabidiol (CBD), cannabinol (CBN), cannabichromene (CBC), cannabigerol (CBG), tetrahydrocannbivarin (THCV), delta 9-tetrahydrocannabinol (A9THC) and many more. It has been shown that Cannabis and its derivatives, alone or in combination with other active agents, can be used for the prevention and/or treatment of chronic pain, multiple sclerosis, multiple sclerosis-related muscle spasms, and pain and/or spasticity in multiple sclerosis, chemotherapy-induced nausea and vomiting (CINV), epilepsy, seizure disorders, and neurological disorders. FDA approved Marinol and Syndros contains delta 9-THC, which currently used in treatment of nausea, vomiting, and anorexia associated with chemotherapy treatments. Furthermore, in April 2016 FDA gave orphan drug designation to cannabidiol for the treatment of Tuberous Sclerosis Complex (TSC), Dravet Syndrome and Lennox-Gastaut Syndrome. Cannabidiol is an orally effective treatment for pain and inflammation.

For example, one of the side-effects most feared by patients undergoing chemotherapy treatment is nausea and vomiting (CINV). In recent years supportive care products to treat the side effects of chemotherapy, such as CINV, have emerged to improve patient comfort and compliance with treatment regimens. Nausea and vomiting patterns are classified as acute or delayed. Approximately 95% of patients receiving chemotherapy will experience some degree of emesis if not prevented with an antiemetic. If untreated, CINV is estimated to affect 85% of cancer patients undergoing chemotherapy and can result in delay or even discontinuation of treatment. And if emesis is not properly managed, it can cause dehydration and poor quality of life, and may eventually lead to interruption or discontinuation of chemotherapy.

CINV patterns are classified as either acute or delayed. Acute onset CINV usually occurs within minutes or a few hours of receiving chemotherapy. The symptoms peak after about 6 hours and can last for approximately 24 hours. Delayed onset CINV is an episode of nausea and vomiting that occurs after 24 hours of administration of chemotherapy and can last for several days. Delayed onset CINV can significantly and adversely affect a patient's ability to provide self-care once the patient has been discharged from the hospital.

Clinicians routinely include antiemetic agents as part of chemotherapy regimens; but according to recent surveys it has been demonstrated that healthcare providers underestimate the frequency and severity of delayed nausea and vomiting after chemotherapy. There is a significant need for a long-acting anti-emetic agent that could be conveniently delivered prior to the administration of chemotherapy and that could prevent both acute and delayed emesis with a single dose, and also providing optimal therapeutic effects while minimizing any undesired side effects resulting from the treatment.

A drug such as Nabilone is recommended for the treatment of nausea and vomiting associated with cancer chemotherapy. Nabilone is a FDA approved drug, comes as an oral capsule (Cesamet). As stated in the Cesamet FDA label₁ “It is indicated for the treatment of nausea and vomiting associated with cancer chemotherapy in patients who have failed to respond adequately to conventional antiemetic treatments”. There is a shortcoming associated with the cesamet oral capsule. Orally cesamet is administered multiple times a day that is two or three times daily. This multiple dosage regimen by oral route is inconvenient for patients who are already experiencing nausea and vomiting.

Additionally, multiple sclerosis (MS) is an autoimmune disease that affects the central nervous system (CNS). The CNS consists of the brain, spinal cord, and the optic nerves. Surrounding and protecting the nerve fibers of the CNS is a fatty tissue called myelin that helps nerve fibers conduct electrical impulses. In MS, myelin is lost in multiple areas, leaving scar tissue called sclerosis. These damaged areas are also known as plaques or lesions. In some cases, the nerve fiber itself is damaged or broken. When myelin or the nerve fiber is destroyed or damaged, the ability of the nerves to conduct electrical impulses to and from the brain is disrupted, and this produces the various symptoms of MS. Patients with MS can expect one of four clinical courses of disease: relapsing-remitting, primary-progressive, secondary-progressive or progressive-relapsing.

Neurodegenerative diseases, such as MS, are a group of disorders characterized by changes in normal neuronal functioning, leading, in most cases, to neuronal death. Most of these diseases are associated, especially in late stages, with severe neuronal loss. With an ever-increasing ageing population, progressively more individuals are affected by neurodegenerative diseases. According to the National Institute of Neurological Disorders and Stroke, there are more than 600 different types of neurological disorders.

Neural degeneration, or neurodegeneration, can be described as the progressive damage or death of neurons. Neurons are nerve cells in the brain whose primary function is to assist in the memory process. The damage or death of neurons leads to a gradual deterioration of the functions controlled by the affected part of the nervous system. Neural degeneration often occurs because of oxidative stress. Oxidative stress occurs to the cells when the effects of pro-oxidants (such as free radicals, reactive oxygen and reactive nitrogen species) exceed the ability of anti-oxidants to neutralize them. When levels of free radicals or other pro-oxidants increase to such an extent, they can cause damage to cell membranes which in turn may result in cell death or damage to genetic material.

Some of the most common types of neurological disorders include Alzheimer's disease, Parkinson's disease and MS. The process of neural degeneration is often the result of glutamate excitotoxicity. Glutamate is a signaling chemical and under normal conditions the concentration of glutamate in a cell tends to be quite low. Glutamate is required at these low concentrations for crucial brain functions such as memory and learning. When glutamate concentrations increase, the process of neural degeneration begins.

Furthermore, pain results from noxious stimulation of nerve endings. Nociceptive pain is caused by noxious stimulation of nociceptors that transmit impulses over intact neural pathways to the spinal neurons and then to the brain. Peripheral neuropathic pain is pain due to damage of the nerve endings, mostly found in the skin, especially in the epidermis. These damaged nerve endings can generate impulses in the absence of stimulation, can be hypersensitive to normal stimulation, and/or can be triggered by remaining local inflammatory stimulation. Even a very small number of damaged and overactive small nerve fibers in the epidermis are sufficient to trigger peripheral neuropathic pain. Neuropathic pain can be debilitating and can reduce quality of life of patients considerably. This pain may persist for months or years beyond the apparent healing of any damaged tissues.

Neuropathic pain has a local inflammatory component that results in sensitization of nerve fibers. Other intact nerve fibers, such as nociceptors being present up in the stratum granulosum, innervating the same region can also be sensitized and participate in clinical symptoms of neuropathic pain (e.g., hyperalgesia). This results in a situation of local neurogenic inflammation resulting in many different clinical features such as burning, freezing, electric shock, itch, tingling, pins and needles, hyperalgesia and allodynia (pain resulting from a non-painful stimulus such as a light touch or stroke). Peripheral nerve damage leads to enhanced transmitter release within the spinal cord and can lead to central sensitization. Increased peripheral input through primary afferents is critically involved in central sensitization and the maintenance of neuropathic pain. Peripherally acting drugs, such as lidocaine 5% medicated patches and capsaicin 8% patches, have demonstrated the ability to reduce pain in neuropathic pain syndromes. However, lidocaine patches are not easy to apply, especially on the toes and by elderly, because the patch has to be cut, and many elderly cannot reach their toes properly. Application of capsaicin creams and patches quite often induce intolerable side effects, such as an increase of burning sensation, and often the treatment has to be combined with a local anesthetic to neutralize this side effect.

In chronic pain in general, for instance, oral analgesics such as acetaminophen, nonsteroidal anti-inflammatory drugs (NSAIDs) and opioids are part of guidelines aimed at to reduce the pain. Chronic use of such oral analgesics however, can induce serious and mortal side effects and/or detrimental drug-drug interactions.

Topical painkiller pharmaceutical compositions are also explored to help patients suffering from chronic pain. Two most commonly used topical compounds in neuropathic pain are capsaicin (vanilloid receptor agonist and counter-irritant) and lidocaine (membrane stabilizer), and both have clear drawbacks.

Several pharmaceutical products exist which contain either phytocannabinoids (natural) or synthetic cannabinoids. For example, dronabinol (Marinol) is the International Nonproprietary Name (INN) for an encapsulated THC product which has been used therapeutically as an appetite stimulant, antiemetic, and analgesic, as an oral drug. Also, nabilone (Cesamet) is a synthetic analog of dronabinol (Marinol), while Sativex is a cannabinoid extract oral spray containing THC, and other cannabinoids that are used to treat neuropathic pain and spasticity. Sativex (combination of THC and cannabidiol) is approved in some countries for the improvement of spasticity symptoms due to multiple sclerosis. Sativex an oromucosal spray is administered multiple times a day that is ranging from one spray/day to 12 sprays/days. Further, rimonabant (marketed under various tradenames) is a selective cannabinoid receptor antagonist used as an anti-obesity drug and as a smoking cessation. Several other cannabinoid-containing products exist.

Thus, considering the therapeutic effect of compounds containing cannabinoids, especially Δ₉-trans-THC, there is a continuing need for improving existing cannabinoid-containing products as well as a need for new products containing cannabinoids, especially in the pharmaceutical field. Clearly, due to multiple oral dosing per day there remains a pressing and long felt need in the art of developing treatment options for multiple sclerosis, multiple sclerosis-related muscle spasms, and pain and/or spasticity in multiple sclerosis in a patient, in particular, for the development of a novel and effective pharmaceutical composition for use in the treatment of chronic pain, the composition inflicting less side effects to the patient.

There is a need for an improved drug delivery system of CBD and/or THC, in combination with outer active agents such as nabilone, for prevention and/or treatment of chronic pain, multiple sclerosis, multiple sclerosis-related muscle spasms, and pain and/or spasticity in multiple sclerosis, chemotherapy-induced nausea and vomiting (CINV), epilepsy, seizure disorders, and neurological disorders which can overcome the drawbacks associated with oral routes that is multiple dosing per day. Transdermal delivery of CBD and/or THC and/or nabilone, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorphs thereof, stereoisomers thereof, coated form thereof, solution thereof in solvents alone or in combinations thereof can address the challenges associated with oral drug delivery, and are useful as treatment of for example, chronic pain, multiple sclerosis, multiple sclerosis-related muscle spasms, and pain and/or spasticity in multiple sclerosis, chemotherapy-induced nausea and vomiting (CINV), epilepsy, seizure disorders, and neurological disorders in a patient.

There is a need for an improved drug delivery system of CBD and/or THC, in combination with outer active agents such as nabilone which can overcome the drawbacks associated with oral routes. Transdermal and/or topical delivery of CBD and/or THC, in combination with outer active agents such as nabilone, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorphs thereof, stereoisomers thereof, coated form thereof, ion-pairs thereof, solution thereof in solvents alone or in combinations thereof can address the challenges associated with oral drug delivery, and are useful as treatment, prevention and/or control of, for example, chronic pain, multiple sclerosis, multiple sclerosis-related muscle spasms, and pain and/or spasticity in multiple sclerosis, chemotherapy-induced nausea and vomiting (CINV), epilepsy, seizure disorders, and neurological disorders.

All references cited herein are incorporated herein by reference in their entireties.

### BRIEF SUMMARY OF THE INVENTION

The Specification provides a pharmaceutical composition comprising an active agent selected from the group consisting of tetrahydrocannabinol (THC), cannabidiol (CBD), nabilone, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorphs thereof, ion-pairs thereof, stereoisomers thereof, racemic forms thereof, and combinations thereof, in a dosage form for transdermal delivery. The Specification provides a pharmaceutical composition which comprises at least about 0.1% to about 80% (w/w) of active agent. The Specification provides a pharmaceutical composition which comprises at least about 0.5-30% of active agent. The Specification provides a pharmaceutical composition which comprises active agent at a concentration of, independently, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%. The Specification provides a pharmaceutical composition formulated as transdermal liquid formulation, transdermal semisolid formulation, transdermal gel formulation, or transdermal polymer matrix formulation, transdermal adhesive matrix formulation, film forming gel, film forming spray formulation, transdermal drug in adhesive matrix formulation. The Specification provides a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, diluents, plasticizers, surfactants, antioxidants, oxidants, and combinations thereof. The Specification provides a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, diluents, plasticizers, surfactants, antioxidants, oxidants, and combinations thereof in the range of 0.1%-99.5% w/w or w/v. The Specification provides a pharmaceutical composition wherein the carrier is present in the range of 5%-99% w/w or w/v. The Specification provides a pharmaceutical composition which is formulated as a transdermal patch. The Specification provides a pharmaceutical composition formulated as a transdermal patch, wherein the transdermal patch is selected from the group such as to reservoir patch, a microreservoir patch, a matrix patch, a pressure sensitive adhesive patch, extended release transdermal film a liquid reservoir system, a microreservoir patch, a matrix patch, a pressure sensitive adhesive patch, a mucoadhesive patch, transdermal film forming formulation.

The Specification provides a pharmaceutical composition indicated for the treatment and/or prevention and/or control of chronic pain in a patient. The Specification provides a pharmaceutical composition which is formulated as the transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, once in 15 days to about 30 days. The Specification provides a pharmaceutical composition which may be formulated as microneedles. The Specification provides a pharmaceutical composition wherein said of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof is produced by a synthetic route.

The Specification provides a method for the treatment and/or prevention and/or control of chronic pain in a patient comprising: selecting a patient in need of treatment and/or prevention and/or control of chronic pain; topically applying the topical pharmaceutical composition as disclosed herein. The Specification provides a method wherein the chronic pain is selected from the group consisting of neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, and any combination thereof. The Specification provides a method wherein the topical application of a transdermal pharmaceutical composition is for the treatment and/or prevention and/or control of chronic pain in a patient, and wherein the transdermal patch is applied at a time period selected from the group consisting of once in a day, once in two days, once in three days, once in four days, once in five days, once in six days, once in a week, and once in ten days, and once in fifteen days, and wherein transdermal film formulation is applied at a time period selected from the group consisting of once in a day, once in 10-20 hours, once in 5-10 hours, once in 5 hours. The Specification provides a method further providing a constant rate of delivery of the active components of the transdermal patch, transdermal film forming formulation over a time period. The Specification provides a method further providing a steady absorption rates of the active components of the transdermal patch, transdermal film forming formulation over a time period. The Specification provides a method further achieving a constant blood serum levels of the active components of the transdermal patch, transdermal film forming formulation over a time period. The Specification provides a method further achieving a reduced variability in dosage of the active components of the transdermal patches, transdermal film forming formulation over a time period. The Specification provides a method further providing a plasma concentration of the active components of the transdermal patch, transdermal film forming formulation in a therapeutic range. The Specification provides a method further providing a plasma concentration of the active components of the transdermal patch, transdermal film forming formulation in a range about 0.01 ng/mL to about 500 ng/mL. The Specification provides a method further providing a plasma concentration of the active components of the transdermal patch, transdermal film forming formulation in a range of about 0.1 ng/mL to about 300 ng/mL. The Specification provides a method for the treatment and/or prevention and/or control of multiple sclerosis, multiple sclerosis related muscle spasms, and pain and/or spasticity in multiple sclerosis in a patient comprising: selecting a patient in need of treatment and/or prevention and/or control of multiple sclerosis, multiple sclerosis-related muscle spasms, pain and/or spasticity in multiple sclerosis; topically applying the transdermal pharmaceutical composition as disclosed herein. The Specification provides a method wherein the topical application of a transdermal pharmaceutical composition for the treatment and/or prevention and/or control of multiple sclerosis, multiple sclerosis-related muscle spasms, pain and/or spasticity in multiple sclerosis in a patient, wherein the transdermal patch is applied at a time period selected from the group consisting of once in a day, once in two days, once in three days, once in four days, once in five days, once in six days, once in a week, and once in ten days, and wherein transdermal film formulation is applied at a time period selected from the group consisting of once in a day, once in 10-20 hours, once in 5-10 hours, once in 5 hours. The Specification provides a method further providing a constant rate of delivery of the active components of the transdermal patch, transdermal film formulation over a time period. The Specification provides a method further providing a steady absorption rates of the active components of the transdermal patch, transdermal film formulation over a time period. The Specification provides a method further achieving a constant blood serum levels of the active components of the transdermal patch, transdermal film formulation over a time period. The Specification provides a method further achieving a reduced variability in dosage of the active components of the transdermal patches, transdermal film formulation over a time period. The Specification provides a method further providing a plasma concentration of the active components of the transdermal patch, transdermal film formulation in a therapeutic range over a period of time. The Specification provides a method further providing a plasma concentration of the active components of the transdermal patch, transdermal film formulation in a therapeutic range of about 0.01 ng/mL to about 500 ng/mL. The Specification provides a method further providing a plasma concentration of the active components of the transdermal patch, transdermal film forming formulation in a range of about 0.1 ng/mL to about 300 ng/mL. The Specification provides a method for the treatment and/or prevention and/or control of chemotherapy-induced nausea and vomiting (CINV) in a patient comprising: selecting a patient in need of treatment and/or prevention and/or control of CINV; topically applying the transdermal pharmaceutical composition as disclosed herein. The Specification provides a method wherein the CINV is selected from the group consisting of acute CINV, delayed CINV, chronic CINV, Breakthrough CNV, refractory CINV, and any combination thereof. The Specification provides a method wherein the topical application of a transdermal pharmaceutical composition is for the treatment and/or prevention and/or control of CINV in a patient, and wherein the transdermal patch is applied at a time period selected from the group consisting of once in a day, once in two days, once in three days, once in four days, once in five days, once in six days, once in a week, and once in ten days. and wherein transdermal film formulation is applied at a time period selected from the group consisting of once in a day, once in 10-20 hours, once in 5-10 hours, once in 5 hours. The Specification provides a method further providing a constant rate of delivery of the active components of the transdermal patch, transdermal film formulation over a time period. The Specification provides a method further providing a steady absorption rates of the active components of the transdermal patch, transdermal film formulation over a time period. The Specification provides a method further achieving a constant blood serum levels of the active components of the transdermal patch, transdermal film formulation over a time period. The Specification provides a method further achieving a reduced variability in dosage of the active components of the transdermal patches, transdermal film formulation over a time period. The Specification provides a method further providing a plasma concentration of the active components of the transdermal patch, transdermal film formulation in a therapeutic range over a period of time.

The Specification provides a method further providing a plasma concentration of the active components of the transdermal patch, transdermal film forming formulation in a range of about 0.01 ng/mL to about 500 ng/mL. The Specification provides a method for the treatment and/or prevention and/or control of epilepsy in a patient comprising: selecting a patient in need of treatment and/or prevention and/or control of epilepsy; topically applying the transdermal pharmaceutical composition as disclosed herein. The Specification provides a method wherein the epilepsy is selected from the group consisting of juvenile myoclonic epilepsy, Lennox-Gastaut, Dravet syndrome, mesial temporal lobe epilepsy, nocturnal frontal lobe epilepsy, progressive epilepsy with mental retardation, progressive myoclonic epilepsy, as well as seizures associated with CNS mass lesions, and any combination thereof. The Specification provides a method wherein the topical application of a transdermal pharmaceutical composition is for the treatment and/or prevention and/or control of epilepsy in a patient, and wherein the transdermal patch is applied at a time period selected from the group consisting of once in a day, once in two days, once in three days, once in four days, once in five days, once in six days, once in a week, and once in ten days, and wherein transdermal film formulation is applied at a time period selected from the group consisting of once in a day, once in 10-20 hours, once in 5-10 hours, once in 5 hours. The Specification provides a method further providing a constant rate of delivery of the active components of the transdermal patch, transdermal film formulation over a time period. The Specification provides a method further providing a steady absorption rates of the active components of the transdermal patch, transdermal film formulation over a time period. The Specification provides a method further achieving a constant blood serum levels of the active components of the transdermal patch, transdermal film formulation over a time period. The Specification provides a method further achieving a reduced variability in dosage of the active components of the transdermal patches, transdermal film formulation over a time period. The Specification provides a method further providing a plasma concentration of the active components of the transdermal patch, transdermal film forming formulation in a therapeutic range over a period of time. The Specification provides a method further providing a plasma concentration of the active components of the transdermal patch, transdermal film forming formulation, in a range of about 0.01 ng/mL to about 500 ng/mL. The Specification provides a method for the treatment and/or prevention and/or control of seizure disorder in a patient comprising: selecting a patient in need of treatment and/or prevention and/or control of seizure disorder; topically applying the transdermal pharmaceutical composition as disclosed herein. The Specification provides a method wherein the seizure disorder is selected from the group consisting of complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, febrile seizures, and any combination thereof. The Specification provides a method wherein the topical application of a transdermal pharmaceutical composition is for the treatment and/or prevention and/or control of seizure disorder in a patient, and wherein the transdermal patch is applied at a time period selected from the group consisting of once in a day, once in two days, once in three days, once in four days, once in five days, once in six days, once in a week, and once in ten days, and wherein transdermal film formulation is applied at a time period selected from the group consisting of once in a day, once in 10-20 hours, once in 5-10 hours, once in hours. The Specification provides a method further providing a constant rate of delivery of the active components of the transdermal patch, transdermal film formulation over a time period. The Specification provides a method further providing a steady absorption rates of the active components of the transdermal patch, transdermal film formulation over a time period. The Specification provides a method further achieving a constant blood serum levels of the active components of the transdermal patch, transdermal film formulation over a time period. The Specification provides a method further achieving a reduced variability in dosage of the active components of the transdermal patches, transdermal film formulation over a time period. The Specification provides a method further providing a plasma concentration of the active components of the transdermal patch, transdermal film forming formulation, in a therapeutic range over a period of time. The Specification provides a method further providing a plasma concentration of the active components of the transdermal patch, transdermal film formulation in a range of about 0.01 ng/mL to about 500 ng/mL. The Specification provides a method for the treatment and/or prevention and/or control of neurological disorder in a patient comprising: selecting a patient in need of treatment and/or prevention and/or control of neurological disorder; topically applying the transdermal pharmaceutical composition as disclosed herein. The Specification provides a method wherein the neurological disorder is selected from the group consisting of cerebral ischemia, ischemia, stroke, neurodegeneration, neurological complications arising from such as Alzheimer's disease, Parkinson's disease, Wilson's disease, Lewy body dementia, multiple sclerosis, seizure disorders, cerebellar ataxia, progressive supranuclear palsy, amyotrophic lateral sclerosis, autism, affective disorders, anxiety disorders, metabolic disorders that affect the CNS, and/or schizophrenia; cell damage; nerve damage from cerebrovascular disorders such as stroke in the brain or spinal cord, from CNS infections including meningitis and HIV, from tumors of the brain and spinal cord, prion diseases, and CNS disorders resulting from ordinary aging (e.g., anosmia), head and/or brain injury, or spinal cord injury, and any combination thereof. The Specification provides a method wherein the topical application of a transdermal pharmaceutical composition is for the treatment and/or prevention and/or control of neurological disorder in a patient, and wherein the transdermal patch is applied at a time period selected from the group consisting of once in a day, once in two days, once in three days, once in four days, once in five days, once in six days, once in a week, and once in ten days, and wherein transdermal film formulation is applied at a time period selected from the group consisting of once in a day, once in 10-20 hours, once in 5-10 hours, once in 5 hours. The Specification provides a method further providing a constant rate of delivery of the active components of the transdermal patch, transdermal film forming formulation over a time period. The Specification provides a method further providing a steady absorption rates of the active components of the transdermal patch over a time period. The Specification provides a method further achieving a constant blood serum levels of the active components of the transdermal patch, transdermal film forming formulation, over a time period. The Specification provides a method further achieving a reduced variability in dosage of the active components of the transdermal patches, transdermal film forming formulation over a time period. The Specification provides a method further providing a plasma concentration of the active components of the transdermal patch, transdermal film forming formulation in a therapeutic range over a period of time. The Specification provides a method further providing a plasma concentration of the active components of the transdermal patch, transdermal film forming formulation in a range of about 0.01 ng/mL to about 500 ng/mL.

### DETAILED DESCRIPTION OF THE INVENTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs. It must be noted that as used herein and in the appended claims, the singular forms “a”, “an”, and “the” include plural referents unless the context clearly dictates otherwise. Thus, for example, reference to “a compound” includes a plurality of compounds.

Unless defined otherwise, all technical and scientific terms used herein have the same meaning as commonly understood by one of ordinary skill in the art to which this invention belongs. As used herein the following terms have the following meanings.

The terms transdermal and topical are used interchangeably.

The term “chronic pain” or “chronic pain states” as used herein, is defined as any pain lasting longer than, for example, about 6 to about 12 weeks.

The term “neuropathic pain” as used herein has its conventional meaning and here means a pain arising as a direct or indirect consequence of a lesion or disease affecting the somatosensory system (central and/or peripheral). Neuropathic pain as used herein, includes all types of neuropathic pain, such as peripheral neuropathy caused by diabetes type 1 or 2, induced by various noxious substances such as alcohol, due to various deficiencies such as vitamin B1, B6 and/or B12 deficiency, various intoxications, such as hypervitaminosis B6, hypothyroidism, chemotherapeutic compound (e.g., paclitaxel or other taxane derivative, vincristine or other vinca alkaloids, cisplatin or other platinum derivate), drug-induced neuropathy, compounds for the treatment of infectious diseases (e.g., streptomycin, didanosine or zalcitabine), or any other chemically toxic compound.

Other peripheral neuropathies include the following: trigeminal neuralgia, post-herpetic neuralgia, intercostal neuralgia, entrapment neuropathy (e.g., carpal tunnel syndrome, tarsal tunnel syndrome, abdominal cutaneous nerve entrapment syndrome), small fiber neuropathy, hereditary motor and sensory neuropathies, chronic inflammatory demyelinating polyneuropathy, sciatic pain chronic idiopathic sensory neuropathy, infectious disease conditions such as post-polio syndrome, AIDS or HIV-associated, Lyme-associated, Sjogren-associated, lymphomatous neuropathy, myelomatous neuropathy, carcinomatous neuropathy, acute pan autonomic neuropathy, vasculitic/ischaemic neuropathy and other mono- and polyneuropathies. Furthermore, under the term “neuropathic pain” also the following is included: complex regional pain syndrome type I and II (reflex sympathetic dystrophy), central neuropathic pain (e.g., thalamic neuropathy, spinal cord injury neuropathy, post stroke pain, multiple sclerosis neuropathy, syringomyelia, spinal cord tumors), phantom limb pain, restless genital syndrome (pain), post-surgical scar pain including cardiac surgery and mastectomy.

The term “inflammatory pain” as used herein has its conventional meaning and here means a pain that arises from inflammation that may be caused but by not limited to trauma, burns, extreme cold, fractures, (osteo)arthritis, rheumatoid arthritis, chronic strains, surgery, infection and autoimmune diseases excessive stretching, infections and vasoconstriction. Multiple inflammatory mediators can directly affect nociceptors or may sensitize them to touch or movement, even some distance from the inflammatory field.

The term “musculoskeletal pain” as used herein has its conventional meaning and here means a pain that affects the muscles, ligaments, tendons, bones, joints and/or soft tissues that are part of the musculoskeletal system. Musculoskeletal pain as used herein, includes all types of pain due to damage of muscle tissue as a result of wear and tear of daily activities. Trauma to an area (jerking movements, auto accidents, falls, sport injuries, fractures, sprains, strains dislocations, and direct blows to the muscle) also can cause musculoskeletal pain. Other causes of musculoskeletal pain include postural strain, repetitive movements, overuse, and prolonged immobilization, misuse of muscles, fibromyalgia, lumbar pain, pain due to increased muscle tone, and tendinitis due to overuse.

The term “treatment” as used herein has its conventional meaning and is here to be considered in its broadest context. The term “treatment” is intended to encompass topical administration of active compounds, i.e., active pharmaceutical ingredients e.g., in a pharmaceutical composition, according to the disclosure, with the aim to alleviate an undesired condition, and therapeutic administration to eliminate or reduce the extent or symptoms of the condition.

Treatment does not necessarily imply that a subject is treated until total recovery.

The term “analgesic” or “analgesics” as used herein has its conventional meaning and here refers to compounds, agents, drugs or substances that reduce pain in its broadest context.

The term “co-analgesic” or “co-analgesics” as used herein has its conventional meaning and here refers to compounds, agents, drugs or substances whose primary indication is for a purpose other than pain relief, which compounds demonstrate analgesic activity.

The term “reinstating analgesic effects” as used herein has its regular scientific meaning and is here referring to the capability (of a compound or of a composition) of reinstating an analgesic effect of at least one analgesic compound or at least one co-analgesic compound, when decreasing analgesic effect occurs after repeated use of a topical formulation containing at least one analgesic or co-analgesic compound.

The term “effect booster” or “co-analgesic effect booster” or “therapeutic effect booster” or “booster effect” or “synergistic effect” as used herein has its conventional meaning and here means the enhancement of a therapeutic effect induced by a co-analgesic compound (“co-analgesic”) leading to 1) intensified therapeutic effects of an active pharmaceutical ingredient with the purpose of alleviating neuropathic pain, inflammatory pain, musculoskeletal pain, pain due to muscle spasms, and/or other chronic pain states, 2) a faster onset of pain relieving effect, 3) a longer duration of analgesia, and/or 4) reinstating analgesic effects, when decreasing analgesic effect occurs after repeated use of a topical pharmaceutical composition containing at least one analgesic compound (“analgesic”) or co-analgesic compound.

“Acute CINV” refers to emesis, nausea and/or vomiting that occurs within the first 24 hours of administration of one or more chemotherapeutic agents to a subject.

“Delayed CINV” refers to emesis, nausea and/or vomiting that occurs more than 24 hours after administration of one or more chemotherapeutic agents to a subject.

“Breakthrough CINV” refers to emesis, vomiting and/or nausea that occur within five days of chemotherapy administration after the use of guideline directed prophylactic antiemetic agents.

“Refractory CINV” refers to emesis, vomiting and/or nausea occurring after chemotherapy in subsequent chemotherapy cycles after guideline directed prophylactic agents have failed in earlier cycles.

The term “topical formulation” as used herein has its conventional meaning and here refers to a formulation that may be applied to skin or mucosa with the aim that a therapeutically active compound penetrates in and/or through the skin, e.g., a topical pharmaceutical composition of the disclosure, e.g., a pharmaceutical composition provided as a topical cream.

As used herein, the term “transdermal delivery” means delivery of drug into systemic circulation through the skin.

As used herein, the term “topical delivery” means delivery of drug to skin for local effect As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human.

In some embodiments, the subject is a non-human animal such as a farm animal (e.g., a horse, pig, or cow) or a pet (e.g., a dog or cat). In a specific embodiment, the subject is a human. As used herein, the term “agent” refers to any molecule, compound, methodology and/or substance for use in the prevention, treatment, management and/or diagnosis of a disease or condition. As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

The term “derivative” or “derivatized” as used herein includes, for example, chemical modification of a compound of the disclosure, or extracted from botanical sources or pharmaceutically acceptable salts thereof or mixtures thereof. That is, a “derivative” may be a functional equivalent of a compound of the disclosure, which is capable of inducing the improved pharmacological functional activity in a given subject.

As used herein, the terms “composition” and “formulation” are used interchangeably.

### Active Agent

The term “active ingredient” refers to an agent, active ingredient compound or other substance, or compositions and mixture thereof that provide some pharmacological, often beneficial, effect. Reference to a specific active ingredient shall include where appropriate the active ingredient and it's pharmaceutically acceptable salts. The disclosure provides for, for example, transdermal formulations and/or topical formulations comprising one or more of the following active agents: Cannabinoids are a group of 21-carbon-containing terpenophenolic compounds produced by Cannabis species. Cannabinoids may also be synthetically produced. The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in Cannabis and some other plants), and synthetic cannabinoids.

Lipophilic cannabinoids are generally grouped as endocannabinoids (most typically as mammalian endocannabinoids); phytocannabinoids, from plant sources; and synthetic cannabinoids. Such cannabinoids are also often classified into the following subclasses: Cannabigerols (CBG); Cannabichromenes (CBC); Cannabidiol (CBD; CBDL); Tetrahydrocannabinol (THC); Cannabinol (CBN); Cannabicyclol (CBL); Cannabielsoin (CBE); and, Cannabitriol (CBT).

Cannabidiol: IUPAC Name 2-[(1R,6R)-6-isopropenyl-3-methylcyclohex-2-en-1-yl]-5-pentylbenzene-1,3-diol Chemical Formula: C₂₁H₃₀O₂ Molecular weight: 314.46 dalton Chemical structure is shown below as formula I

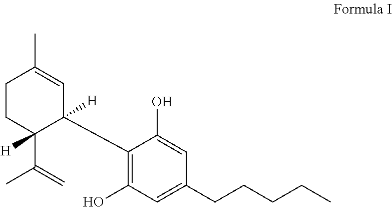 [SMILES: C=C(C)[C@@H]1CCC(C)=C[C@H]1c1c(O)cc(CCCCC)cc1O]

Tetrahydrocannbinol (THC): IUPAC Name (−)-(6aR,10aR)-6,6,9-Trimethyl-3-pentyl-6a,7,8,10a-tetrahydro-6H-benzo[c]chromen-1-ol Chemical Formula: C₂₁H₃₀O₂
Molecular weight: 314.47 dalton.
Chemical structure is shown below as formula II

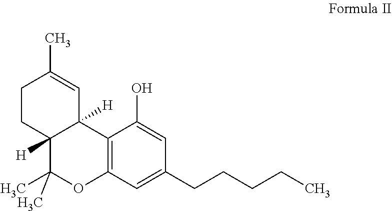 [SMILES: CCCCCc1cc(O)c2c(c1)OC(C)(C)[C@@H]1CCC(C)=C[C@@H]21]

As used herein, the word Cannabis refers to all pharmaceutically acceptable forms of Cannabis and its derivatives either alone or in combinations thereof, for example, in following forms but not limited to such as free base or salts or isomers or amorphous or crystalline or co crystalline or solid solution or prodrugs or analogs or derivatives or metabolites or polymorphs or its stereoisomer or coated form or ion-pairs. For example, cannabidiol's free base or its salts or its isomers or its amorphous form or its crystalline form or its co crystalline form or its solid solution or its prodrugs or its analogs or its derivatives or synthetic forms or its polymorphs or its stereoisomer or its ion-pairs. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, ptoluenesulphonate and pamoate salts. Suitable base salts are formed from bases which form nontoxic salts and examples are the sodium, potassium, aluminium, calcium, magnesium, zinc and diethanolamine salts.

As used herein, the term “cannabidiol” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorph thereof, stereoisomers thereof, powder form thereof, liquid form thereof, ion-pairs thereof, solution of cannabidiol in solvents such as but not limited to methanol, etc., alone or in combinations thereof. In certain embodiments the CBD is 100% synthetic. In certain embodiments the CBD has a purity equal to or greater than about 90%, 91%, 92%, 93%, 94%, 95%, 96%, 97%, 98%, 99%, 99.5%, 99.75%, or 100% (w/w). In certain embodiments the CBD is produced synthetically and has a purity equal to or greater than about 90%, 91%, 92%, 93%, 94%, 95%, 96%, 97%, 98%, 99%, 99.5%, 99.75%, or 100% (w/w).

As used herein, the term “THC” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, powder form thereof, liquid form thereof, ion-pairs thereof, polymorph thereof, stereoisomers thereof, solution of THC in solvents such as but not limited to methanol, heptane, ethanol, etc. alone or in combinations thereof. In certain embodiments the THC agent is 100% synthetic. In certain embodiments the THC has a purity equal to or greater than about 90%, 91%, 92%, 93%, 94%, 95%, 96%, 97%, 98%, 99%, 99.5%, 99.75%, or 100% (w/w). In certain embodiments the THC is produced synthetically and has a purity equal to or greater than about 90%, 91%, 92%, 93%, 94%, 95%, 96%, 97%, 98%, 99%, 99.5%, 99.75%, or 100% (w/w).

As used herein, synthetic cannabinoids include at least the following: AM-087 is an analgesic drug that is a cannabinoid agonist derivative of Δ8THC substituted on the 3-position side chain and a potent CB1 agonist; AM-251 is an inverse agonist at the CB1 cannabinoid receptor with close structural similarity to SR141716A (rimonabant), both of which are biarylpyrazole cannabinoid receptor antagonists as well as μ-opioid receptor antagonist; Methanandamide (AM-356) is a stable chiral analog of anandamide and acts on the cannabinoid receptors with a Ki of 17.9 nM at CB1 and 868 nM at CB2; AM-374—palmitylsulfonyl fluoride; AM-381—stearylsulfonyl fluoride; AM404, also known as N-arachidonoylaminophenol, is an active metabolite of paracetamol (acetaminophen) thought to induce its analgesic action through its activity on the endocannabinoid, COX, and TRPV systems, all of which are present in pain and thermoregulatory pathways; AM-411 is an analgesic that is a cannabinoid agonist; AM-411 is a potent and fairly selective CB1 full agonist and produces similar effects to other cannabinoid agonists such as analgesia, sedation, and anxiolysis; AM-630 (6-lodopravadoline) acts as a potent and selective inverse agonist for the cannabinoid receptor CB2, selectivity over CB1 where it acts as a weak partial agonist; AM-661—1-(N-methyl-2-piperidine)methyl-2-methyl-3-(2-iodo)benzoylindole; JWH-018 (1-pentyl-3-(1-naphthoyl)indole) or AM-678 is an analgesic chemical from the naphthoylindole family that acts as a full agonist at both the CB1 and CB2 cannabinoid receptors, with some selectivity for CB2; AM-679 acts as a moderately potent agonist for the cannabinoid receptors; AM-694 (1-(5-fluoropentyl)-3-(2-iodobenzoyl)indole) acts as a potent and selective agonist for the cannabinoid receptor CB1; AM-735—3-bornyl-Δ8-THC, a mixed CB1/CB2 agonist; AM-855 is an analgesic cannabinoid agonist at both CB1 and CB2 with moderate selectivity for CB1; AM-881—a chlorine-substituted stereoisomer of anandamide whose Ki=5.3 nM at CB1 and 95 nM at CB2; AM-883 an allyl-substituted stereoisomer of anandamide whose Ki=9.9 nM at CB1 and 226 nM at CB2; AM-905 is an analgesic cannabinoid which acts as a potent and reasonably selective agonist for the CB1 cannabinoid receptor; AM-906 is an analgesic drug which is a cannabinoid agonist and is a potent and selective agonist for the CB1 cannabinoid receptor; AM-919 is an analgesic cannabinoid receptor agonist, potent with respect to both CB1 and CB2; AM-926—a potent agonist at both CB1 and CB2 with moderate selectivity for CB1; AM-938 is an analgesic drug which is a cannabinoid receptor agonist and while it is still a potent agonist at both CB1 and CB2, it is reasonably selective for CB2; AM-1116—a dimethylated stereoisomer of anandamide; AM-1172—an endocannabinoid analog specifically designed to be a potent and selective inhibitor of AEA uptake that is resistant to FAAH hydrolysis; AM-1220 is a potent and moderately selective agonist for the cannabinoid receptor CB1; AM-1221 acts as a potent and selective agonist for the cannabinoid receptor CB2; AM-1235 (1-(5-fluoropentyl)-3-(naphthalen-1-oyl)-6-nitroindole) acts as a potent and reasonably selective agonist for the cannabinoid receptor CB1; AM-1241 (1-(methylpiperidin-2-ylmethyl)-3-(2-iodo-5-nitrobenzoyl)indole) is a potent and selective agonist for the cannabinoid receptor CB2, with analgesic effects in mammals, particularly against “atypical” pain such as hyperalgesia and allodynia, and has also shown efficacy in the treatment of amyotrophic lateral sclerosis in mammalian models; AM-1248 acts as a moderately potent agonist for both the cannabinoid receptors CB1 and CB2; AM-1710—a CB2 selective cannabilactone with 54× selectivity over CB1; AM-1714 acts as a reasonably selective agonist of the peripheral cannabinoid receptor CB2 and has both analgesic and anti-allodynia effects; AM—2201 (1-(5-fluoropentyl)-3-(1-naphthoyl)indole) acts as a potent but nonselective full agonist for the cannabinoid receptor; AM-2212—a potent agonist at both CB1 and CB2; AM-2213—a potent agonist at both CB1 and CB2; AM-2232 (1-(4-cyanobutyl)-3-(naphthalen-1-oyl)indole) acts as a potent but unselective agonist for the cannabinoid receptors CB1 and CB2; AM-2233 acts as a highly potent full agonist for the cannabinoid receptors CB1 and CB2 and has been found to fully substitute for THC in certain mammalian studies, with a potency lower than that of JWH-018 but higher than WIN 55,212-2; AM-2389 acts as a potent and reasonably selective agonist for the CB1 receptor; AM-3102—an analog of oleoylethanolamide, (the endogenous agonist for proliferator activated receptor α (PPAR α)) it acts as a weak cannabinoid agonist at CB1 and at CB2; AM-4030 an analgesic which is potent agonist at both CB1 and CB2, but also reasonably selective for CB1; AM-4054 is a potent but slow-onset agonist with CB1 affinity and selectivity CB1 over CB2; AM-4113—a CB1 selective neutral antagonist; AM-6545 acts as a peripherally selective silent antagonist for the CB1 and was developed for the treatment of obesity; JWH-007—an analgesic which acts as a cannabinoid agonist at both the CB1 receptor and CB2 receptors, with some selectivity for CB2, JWH-007 is an analgesic which acts as a cannabinoid agonist at both the CB1 and CB2 receptors; JWH-015 acts as a subtype-selective cannabinoid agonist which binds almost 28× more strongly to CB2 than CB1. and has been shown to have immunomodulatory effects, and may be useful in the treatment of pain and inflammation; JWH-018 an analgesic which acts as a full agonist at both the CB1 and CB2 cannabinoid receptors and produces effects similar to those of THC; JWH-019—an agonist at both CB1 and CB2 receptors and is an analgesic from the naphthoylindole family that acts as a cannabinoid agonist at both the CB1 and CB2 receptors; JWH-030—an analgesic which is a partial agonist at CB1 receptors; JWH-047—a potent and selective agonist for the CB2 receptor, JWH-048—a potent and selective agonist for the CB2 receptor, JWH-051—an analgesic with a high affinity for the CB1 receptor, but is a much stronger agonist for CB2, JWH-057—a 1-deoxy analog of Δ8-THC that has very high affinity for the CB2 receptor, but also has high affinity for the CB1 receptor; JWH-073—an analgesic which acts as a cannabinoid agonist at both the CB1 and CB2 receptors. It is somewhat selective for the CB1 subtype; JWH-081—an analgesic which acts as an agonist at both the cannabinoid CB1 AND CB2 receptors; JWH-098—a potent and fairly selective CB2 agonist; JWH-116—a CB1 ligand; JWH-120—a potent and 173-fold selective CB2 agonist; JWH-122—a potent and fairly selective CB1 agonist; JWH-133—a potent and highly selective CB2 receptor agonist; 139—3-(1,1-dimethylpropyl)-6,6,9-trimethyl-6a,7,10,10a-tetrahydro-6H-benzo[c]chromene; JWH-147—an analgesic from the naphthoylpyrrole family, which acts as a cannabinoid agonist at both the CB1 and CB2 receptors; JWH-148—a moderately selective ligand for the CB2 receptor, with more than 8 times selectivity over the CB1 subtype; JWH-149—a potent and fairly selective CB2 agonist; JWH-161—a CB1 ligand; JWH-164—a potent cannabinoid agonist; JWH-166—a potent and highly selective CB2 agonist; JWH-167—a weak cannabinoid agonist from the phenylacetylindole family; JWH-171—an analgesic which acts as a cannabinoid receptor agonist; JWH-175—(1-pentylindol-3-yl)naphthalen-1-ylmethane, 22 nM at CB1, JWH-176—1-([(1E)-3-pentylinden-1-ylidine]methyl)naphthalene; JWH-181—a potent cannabinoid agonist; JWH-182—a potent cannabinoid agonist with some selectivity for CB1; JWH-184—1-pentyl-1H-indol-3-yl-(4-methyl-1-naphthyl)methane; JWH-185—1-pentyl-1H-indol-3-yl-(4-methoxy-1-naphthyl)methane; JWH-192—(1-(2-morpholin-4-yl ethyl)indol-3-yl)-4-methylnaphthalen-1-ylmethane; JWH-193—(1-(2-morpholin-4-ylethyl)indol-3-yl)-4-methylnaphthalen-1-ylmethanone; JWH-194—2-methyl-1-pentyl-1H-indol-3-yl-(4-methyl-1-naphthyl)methane; JWH-195—(1-(2-morpholin-4-yl ethyl)indol-3-yl)-naphthalen-1-yl methane; JWH-196—2-methyl-3-(1-naphthalenylmethyl)-1-pentyl-1H-Indole; JWH-197—2-methyl-1-pentyl-1H-indol-3-yl-(4-methoxy-1-naphthyl)methane; JWH-198—(1-(2-morpholin-4-ylethyl)indol-3-yl)-4-methoxynaphthalen-1-ylmethanone; JWH-199—(1-(2-morpholin-4-ylethyl)indol-3-yl)-4-methoxynaphthalen-1-ylmethane; JWH-200—an analgesic from the aminoalkylindole family, which acts as a cannabinoid receptor agonist; JWH-203—an analgesic from the phenylacetylindole family, which acts as a cannabinoid agonist with approximately equal affinity at both the CB1 and CB2 receptors; JWH-205—142-methyl-1-pentylindol-3-yl)-2-phenylethanone; JWH-210—an analgesic chemical from the naphthoylindole family, which acts as a potent cannabinoid agonist at both the CB1 and CB2 receptors; JWH-213—a potent and fairly selective CB2 agonist; JWH-229—1-methoxy-3-(1′,1′-dimethylhexyl)-48-THC, a dibenzopyran cannabinoid which is a potent CB2 agonist; JWH-234—a cannabinoid agonist with selectivity for CB2; JWH-250—an analgesic from the phenylacetylindole family, which acts as a cannabinoid agonist at both the CB1 and CB2 receptors; JWH-251—(1-pentyl-3-(2-methylphenylacetyl)indole); JWH-258—a potent and mildly selective CB1 agonist; JWH-302—(1-pentyl-3-(3-methoxyphenylacetyl)indole); JWH-307—an analgesic from the naphthoylpyrrole family, which acts as a cannabinoid agonist at both the CB1 and CB2 receptors that is somewhat selective for the CB2 subtype; JWH-350—a 11-nor-1-methoxy-3-(1′,1′-dimethylheptyl)-9α-hydroxyhexahydrocannabinol has a 33-fold selectivity for the CB2 receptor and high CB2 receptor affinity with little affinity for the CB1 receptor; JWH-359—a dibenzopyran cannabinoid that is a potent and selective CB2 receptor agonist; JWH-387—1-pentyl-3-(4-bromo-1-naphthoyl)indole, an analgesic from the naphthoylindole family, which acts as a potent cannabinoid agonist at both receptors CB1 and CB2; JWH-398—an analgesic chemical from the naphthoylindole family, which acts as a potent cannabinoid agonist at both receptors with a Ki of 2.3 nM at CB1 and 2.8 nM at CB2; JWH-424—a potent and moderately selective CB2 agonist with a Ki of 5.44 nM at CB2 and 20.9 nM at CB1; HU-210 is a cannabinoid that is 100 to 800 times more potent than natural THC from Cannabis and has an extended duration of action and is a potent analgesic with many of the same effects as natural THC; Ajulemic acid (AB-III-56, HU-239, IP-751, CPL 7075, CT-3, Resunab) is a cannabinoid derivative of the non-psychoactive THC metabolite 11-nor-9-carboxy-THC that shows useful analgesic and anti-inflammatory effects without causing a subjective “high”. It is being developed for the treatment of neuropathic pain and inflammatory conditions such as arthritis and for the treatment of orphan life-threatening inflammatory diseases; HU-243 (AM-4056) is a cannabinoid which is a potent agonist at both the CB1 and CB2 receptors; HU-308 acts as a cannabinoid agonist and is highly selective for the CB2 receptor subtype. It has analgesic effects, promotes proliferation of neural stem cells, and protects both liver and blood vessel tissues against oxidative stress via inhibition of TNF-α; HU-331 is a quinone anticarcinogenic synthesized from cannabidiol; HU-336 is a strongly antiangiogenic compound, it inhibits angiogenesis by directly inducing apoptosis of vascular endothelial cells without changing the expression of pro- and antiangiogenic cytokines and their receptors; HU-345 (cannabinol quinone) is a drug that is able to inhibit aortic ring angiogenesis more potently than its parent compound cannabinol; CP 47,497 or (C7)-CP 47,497 is a cannabinoid receptor agonist drug.

The disclosure also provides methods for the biosynthesis of cannabinoids and for the use of a eukaryotic or prokaryotic expression system for the production of biosynthetic enzymes that can be used for the manufacture of cannabinoids and cannabinoid analogs. Yeast as well as eukaryotic and prokaryotic cells are suitable for the cloning and expression of the cannabinoid acid synthase enzymes and include without limitation E coli, yeast and baculovirus hosts. Thus, the present disclosure provides a method for the production of biosynthetic cannabinoids, such as for example THC and/or CBD, using cannabinoid acid synthase enzymes including, but not limited to, tetrahydrocannabinolic acid (THCA) synthase and cannabidiolic acid (CBDA) synthase. The disclosure further provides for the transdermal and/or topical compositions as disclosed herein comprising, for example, biosynthetic CBD, alone or in combination with other active agents.

According to certain embodiments, transdermal and/or topical compositions described herein are for the prevention and/or treatment of pain and/or inflammation. According to certain embodiments, transdermal and/or topical compositions described herein are for the reduction in severity of pain and/or inflammation.

Purified CBD: The disclosure provides that the CBD is present in an amount that reduces total seizure frequency by greater than 70% with respect to the seizure frequency achieved on concomitant anti-epileptic drugs (AED). More preferably the CBD is present in an amount that reduces total seizure frequency by greater than 90% with respect to the seizure frequency achieved on concomitant anti-epileptic drugs (AED). More preferably still the CBD is present in an amount that reduces total seizure frequency by 100% with respect to the seizure frequency achieved on concomitant anti-epileptic drugs (AED).

In one embodiment the CBD is present as a highly purified extract of Cannabis which comprises at least 90%, 91%, 92%, 93%, 94%, 95%, 96%, 97%, 98%, 99%, 99.5%, or 99.75% (w/w) CBD. In certain embodiments the CBD is 100% synthetic. In certain embodiments the CBD has a purity equal to or greater than about 90%, 91%, 92%, 93%, 94%, 95%, 96%, 97%, 98%, 99%, 99.5%, 99.75%, or 100% (w/w). In certain embodiments the CBD is produced synthetically and has a purity equal to or greater than about 90%, 91%, 92%, 93%, 94%, 95%, 96%, 97%, 98%, 99%, 99.5%, 99.75%, or 100% (w/w). The one or more AED is preferably selected from the group consisting of: clobazam; levetiracetam; topiramate; stiripentol; phenobarbital; lacsamide; valproic acid; zonisamide; perampanel; and fosphenytoin. In certain embodiments the CBD is used in combination with clobazam. Preferably the number of different anti-epileptic drugs or the dose of AED that are used in combination with the CBD is reduced. More preferably the dose of AED which is reduced is of clobazam.

In certain embodiments, the dose of CBD is greater than, for example, about 0.5, 1, 2, 3, 4, 5, 6, 7, 8, 9, 10, 15, 20, 25, 30, 35, 40, or 45 mg/kg/day. For example, a for a 15 kg patient a dose of greater than 75 mg of CBD per day would be provided. Doses greater than 5 mg/kg/day such as greater than 10/mg/kg/day, greater than 15 mg/kg/day, greater than 20 mg/kg/day and greater than 25 mg/kg/day are also envisaged to be effective. In certain embodiments, the dose of CBD is greater than, for example, about 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, or 275 mg/day.

The disclosure provides a method of treating treatment-resistant epilepsy comprising administering cannabidiol (CBD) to a subject, wherein the epilepsy is febrile infection related epilepsy syndrome (FIRES).

The disclosure provides a method of treating treatment-resistant epilepsy comprising administering cannabidiol (CBD) to a subject in an amount sufficient to reduce total seizure frequency by greater than 50% with respect to the seizure frequency achieved on one or more concomitant anti-epileptic drugs (AED).

Nabilone: Chemical Name: (±)-trans-3-(1,1-dimethylheptyl)-6,6a,7,8,10,10a-hexahydro-lhydroxy-6-6-dimethyl-9H-dibenzo[b,d]pyran-9-one
Empirical Formula: C₂₄H₃₆O₃
Molecular weight: 372.55

### Structure: Formula III

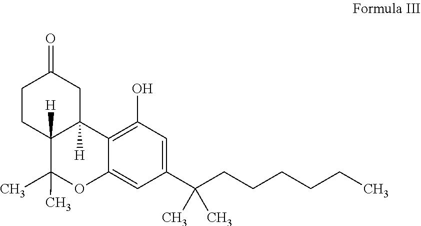

Nabilone is white to off—white polymorphic crystalline powder₁.

In embodiments as disclosed herein, without any limitation preferable form of Nabilone is selected from group such as nabilone polymorphic crystalline powder, nabilone co crystals, nabilone amorphous form, coated nabilone, nabilone crystalline form, nabilone polymorphs, nabilone salt, nabilone's racemic forms, nabilone's ion-pairs, or nabilone's isomers. Preferred form is Nabilone polymorphic crystalline powder.

Amorphous form of the drug does not have a definite structure. Amorphous form of drug has higher solubility as compared to crystalline forms. Different techniques and methods are used to make amorphous form of drugs.

As stated in regulatory classification of pharmaceutical Co-Crystals guidance for Industry “Co-crystals are crystalline materials composed of two or more different molecules, typically drug and co-crystal formers (“coformers”), in the same crystal lattice”2. Different methods are available for the preparation of Cocrystals. Each drug has distinct chemical structure and physicochemical properties therefore, it is difficult to predict the success rate of cocrystallization reaction. A lot of experiments are required with different experimental conditions to see whether cocrystals of drug can be prepared.

Coating of the drug can be done with polymer or other excipients. Different techniques are used for the coating of drug. Stability of the drug can also be increased by encapsulation.

As used herein the term “combination administration” of a compound, therapeutic agent or known drug with the combination of the present disclosure means administration of the drug and the one or more compounds at such time that both the known drug and/or combination will have a therapeutic effect. In some cases this therapeutic effect will be synergistic. Such concomitant administration can involve concurrent (i.e. at the same time), prior, or subsequent administration of the drug with respect to the administration of the composition and/or combination of the present disclosure.

A person of ordinary skill in the art would have no difficulty determining the appropriate timing, sequence and dosages of administration for particular drugs of the present disclosure.

Further, active ingredient(s), where applicable, may be present either in the form of one substantially optically pure enantiomer or as a mixture of enantiomers or polymorphs thereof. The active ingredient(s) may comprise one or more of the following therapeutic classes but not limited to adrenergic agent; adrenocortical steroid; adrenocortical suppressant; aldosterone antagonist; amino acid; anabolic; analeptic; analgesic; anesthetic; anorectic; anti-acne agent; antiadrenergic; anti-allergic; anti-amebic; anti-anemic; anti-anginal; anti-arthritic; anti-asthmatic; antiatherosclerotic; antibacterial; anticholinergic; anticoagulant; anticonvulsant; antidepressant; antidiabetic; antidiarrheal; antidiuretic; anti-emetic; anti-epileptic; antifibrinolytic; antifungal; antihemorrhagic; antihistamine; antihyperlipidemia; antihypertensive; antihypotensive; antiinfective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; anti-ulcerative; antiviral; appetite suppressant; blood glucose regulator; bone resorption inhibitor; bronchodilator; cardiovascular agent; cholinergic; depressant; diagnostic aid; diuretic; dopaminergic agent; estrogen receptor agonist; fibrinolytic; fluorescent agent; free oxygen radical scavenger; gastric acid supressant; gastrointestinal motility effector; glucocorticoid; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; psychotropic; radioactive agent; scabicide; sclerosing agent; sedative; sedative-hypnotic; selective adenosine Al antagonist; serotonin antagonist; serotonin inhibitor; serotonin receptor antagonist; steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; xanthine oxidase inhibitor. Examples of active ingredients comprises, but is not limited to any of the following, for example, alone or in combination: Examples of active ingredients comprise drugs which are administered for the treatment and/or prevention and/or control and/or management of symptoms associated with neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia alone or in combinations thereof.

In one aspect examples of active ingredients comprise drugs such as but not limited to lidocaine, capsaicin, Tricyclic Antidepressants (not limited to such as amitriptyline, imipramine, nortriptyline, desipramine, doxepin, etc.), SNRIs and SSRIs (not limited to such as duloxetine, venlafaxine, fluoxetine, milnacipran etc.), NSAIDS (not limited to diclofenac, aspirin, naproxen, ibuprofen, ketoprofen, celecoxib, meloxicam, etc.), acetaminophen, cox-2 inhibitors (not limited to celecoxib, etc.), anticonvulsants (not limited to such as carbamazepine, gabapentin, lamotrigine, pregabalin, oxcarbazepine, lamotrigine, etc.), valproic acid, menthol, camphor, methyl salicylate, salicylates, corticosteroid drugs (not limited to such as triamcinolone, methylprednisolone, cortisone, prednisone, dexamethasone, etc), opioid, etc.

In one aspect transdermal delivery system and/or topical delivery system comprising drug combination of two or more drugs such as but not limited to THC, CBD, lidocaine, menthol, capsaicin, methyl salicylate, etc. Examples of drug combination of two or more drugs for transdermal delivery systems and/or topical delivery system includes such as but not limited to THC, CBD, lidocaine and combinations thereof, THC, CBD, menthol and combinations thereof, THC, CBD, capsaicin and combinations thereof, THC, CBD, methyl salicylate and combinations thereof, etc.

In another aspect transdermal delivery system and/or topical delivery system comprising drug combination of two or more drugs such as but not limited to THC, CBD, antidepressant drug, NSAIDS, anticonvulsants drug, corticosteroid drug, pain relievers, etc. Examples of drug combination of two or more drugs for transdermal delivery systems and/or topical delivery system includes such as but not limited to THC, CBD, antidepressant drug and combinations thereof, THC, CBD and anticonvulsant drug and combinations thereof, THC, CBD, corticosteroid drug and combinations thereof, THC, CBD, NSAID drug and combinations thereof, etc.

In yet another aspect transdermal delivery system and/or topical delivery system comprising drug combination of one or more drugs such as but not limited to nabilone and cannabinoids. In another aspect transdermal delivery system and/or topical delivery system comprising nabilone for treatment of chemotherapy induced nausea and vomiting As indicated the pharmaceutical formulations as disclosed herein may comprise auxiliary excipients such as for example diluents, binders, lubricants, surfactants, disintegrants, plasticisers, tackifiers, opacifying agents, pigments, polymers, solvents, and such like. As will be appreciated by those skilled in the art, the exact choice of excipient and their relative amounts will depend to some extent on the final transdermal or topical dosage form.

According to certain embodiments described herein, pharmaceutical composition or transdermal formulation or topical formulation contains, active agent(s) as set forth herein, such as, cannabidiol and/or THC—the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, stereoisomers thereof, coated form thereof, solid solution thereof, ion-pairs thereof, solution thereof in solvents alone or in combinations thereof. More preferably transdermal and topical formulation may include cannabidiol, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, ion-pairs thereof, stereoisomers thereof, coated form thereof, solution of cannabidiol in methanol, alone or in combinations thereof. More preferably transdermal and topical formulation may include THC, the free base thereof, salts thereof, isomers thereof, amorphous forms thereof, crystalline forms thereof, co crystalline forms thereof, prodrugs thereof, analogs thereof, derivatives thereof, ion-pairs thereof, synthetic forms thereof, biosynthetic forms thereof, active metabolites thereof, polymorphs thereof, stereoisomers thereof, coated form thereof, solution of cannabidiol in methanol, solution of cannabidiol in ethanol alone or in combinations thereof. According to certain embodiments described herein, pharmaceutical composition or transdermal formulation or topical formulation contains, active agent(s) as set forth herein, such as, nabilone free base thereof, salts thereof, isomers thereof, amorphous forms thereof, crystalline forms thereof, co crystalline forms thereof, prodrugs thereof, analogs thereof, derivatives thereof, synthetic forms thereof, active metabolites thereof, polymorphs thereof, stereoisomers thereof, coated form thereof, solid solution thereof, ion-pairs thereof, solution thereof in solvents alone or in combinations thereof. More preferably transdermal and topical formulation may include nabilone in polymorphic crystalline powder.

As used herein, the word active agent refers to all pharmaceutically acceptable forms of the active agent and its derivatives either alone or in combinations thereof, for example, in following forms but not limited to such as free base or salts or isomers or amorphous or crystalline or co crystalline or solid solution or prodrugs or analogs or derivatives or metabolites polymorphs thereof, stereoisomers thereof, coated form thereof, ion-pairs thereof. For example, the active agent's free base or its salts or its isomers or its amorphous form or its crystalline form or its co crystalline form or its solid solution or its prodrugs or its analogs or its derivatives or synthetic forms polymorphs thereof, ion-pairs thereof, stereoisomers thereof, coated form thereof. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts. The active ingredient(s) can be present in the form of a free base or in the form of pharmaceutically acceptable salts. Pharmaceutically acceptable salts forming part of this disclosure are intended to define but not limited to salts of the carboxylic acid moiety such as alkali metal salts like Li, Na and K salts; alkaline earth metal salts like Ca and Mg salts; salts of organic bases such as lysine, arginine, guanidine, diethanolamine, choline, and the like; ammonium or substituted ammonium salts and aluminium salts. Salts may be acid addition salts which defines but not limited to sulfates, nitrates, phosphates, perchlorates, borates, hydrohalides, acetates, tartrates, maleates, citrates, succinates, palmoates, methanesulfonates, benzoates, salicylates, hydroxynaphthoates, benzensulfonates, ascorbates, glycerophosphates, ketoglutarates and the like.

As used herein, the term active agent includes the free base thereof, salts thereof, isomers thereof, amorphous forms thereof, crystalline forms thereof, co crystalline forms thereof, prodrugs thereof, analogs thereof, derivatives thereof, synthetic forms thereof, polymorphs thereof, stereoisomers thereof, coated form thereof, ion-pairs thereof, racemic forms thereof, natural extracts thereof, biosynthetic forms thereof, alone or in combinations thereof. In certain embodiments the active agent is highly purified. In certain embodiments the active agent is present as a highly purified extract of active agent which comprises at least 90%, 91%, 92%, 93%, 94%, 95%, 96%, 97%, 98%, 99%, 99.5%, or 99.75% (w/w) of the formulation. In certain embodiments the active agent is provided at a concentration equal to or greater than about 90%, 91%, 92%, 93%, 94%, 95%, 96%, 97%, 98%, 99%, 99.5%, 99.75%, or 100% (w/w).

In certain embodiments the active agent is 100% synthetic. In certain embodiments the active agent has a purity equal to or greater than about 90%, 91%, 92%, 93%, 94%, 95%, 96%, 97%, 98%, 99%, 99.5%, 99.75%, or 100% (w/w). In certain embodiments the active agent is produced synthetically and has a purity equal to or greater than about 90%, 91%, 92%, 93%, 94%, 95%, 96%, 97%, 98%, 99%, 99.5%, 99.75%, or 100% (w/w). In certain embodiments the active agent is a combination of active agents, and each active agent may be produced synthetically and independently have a purity equal to or greater than about 90%, 91%, 92%, 93%, 94%, 95%, 96%, 97%, 98%, 99%, 99.5%, 99.75%, or 100% (w/w).

In certain embodiments, the dose of active agent is equal to or greater than, for example, independently, about 0.01, 0.1, 0.25, 0.5, 1, 2, 3, 4, 5, 6, 7, 8, 9, 10, 15, 20, 25, 30, 35, 40, or 45 mg/kg/day. In certain embodiments, the active agent is titrated from 5 to 20-25 mg/kg/day and optionally maintained for 10-15 days. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 microgram/day. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 mg/day. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 microgram. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 mg. In exemplary embodiments, formulations of the disclosure may comprise active agent at a concentration of, independently, about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% of the formulation. In exemplary embodiments, formulations of the disclosure may comprise active agent at a concentration, independently, of about 0.1 to about 50%, about 1 to 20%, of about 5% to 25%, about 10% to about 20%, or about 15% to about 18%, about 30% to about 70%, about 35% to about 65%, about 63.13%, about 40% to about 64% w/w, about 95 to about 98%, or about 95 to about 97%. In exemplary formulations of the disclosure, the active agent will represent approximately about 0.1 wt % to about 50% wt %, 1 wt % to 75 wt %, preferably 2 wt % to 30 wt %, more preferably 5 wt. % to 20 wt. % of the formulation.

As used herein, the term “pharmaceutically acceptable salts” includes acid addition salts or addition salts of free bases. The term “pharmaceutically acceptable salts” of the active agent within its scope all the possible isomers and their mixtures, and any pharmaceutically acceptable metabolite, bioprecursor and/or pro-drug, such as, for example, a compound which has a structural formula different from the one of the compounds of the disclosure, and yet is directly or indirectly converted in vivo into a compound of the disclosure, upon administration to a subject, such as a mammal, particularly a human being.

As used herein, the term “transdermal delivery” means delivery of drug into systemic circulation through the skin.

### Pharmaceutical Compositions

According to certain embodiments described herein, pharmaceutical composition or transdermal formulation of contains active agents such as THC and/or CBD and/or nabilone, and derivatives of these compounds.

One embodiment of the present disclosure can be a transdermal drug delivery system which may include without any limitation to transdermal formulation, transdermal patches, topical formulation, microneedles, iontophoresis, metered dose transdermal spray, metered dose transdermal gel, transdermal film forming formulations, transdermal aerosols.

Transdermal formulation which includes liquids for example without any limitation like solutions, suspensions, dispersions, emulsion. Transdermal formulation includes semisolids for example without any limitations like gels, ointments, emulsions, creams, suspension, paste, lotion, balm. Liquid formulation and/or gel formulation incorporated in transdermal patch, metered dose transdermal system, sachet, etc. Transdermal formulations which includes matrix patches without any limitations like adhesive matrix patch, drug in adhesive matrix patch, non-adhesive matrix patch, a transdermal matrix formulation as drug in adhesive matrix patch is preferred. Other transdermal formulations include transdermal gel, transdermal meter dose spray, transdermal meter dose aerosols, transdermal film forming formulations such as but not limited to transdermal meter dose film forming spray, transdermal meter dose film forming gel, transdermal film forming gel, transdermal film forming spray.

Without any limitation, transdermal patch may include all transdermal drug delivery systems stated in art preferably but not limited to reservoir patch, matrix patch, bilayer matrix patch, multilayer matrix patch, microreservoir patch, adhesive systems, transdermally applicable tape and other. In certain embodiments of the present disclosure, a transdermal patch comprises transdermal formulation containing active agents such as THC and/or CBD and/or nabilone, and derivatives of these compounds contained in a reservoir or a matrix, and an adhesive which allows the transdermal patch to adhere to the skin, allowing the passage of the active agents from the transdermal patch through the skin of the patient. The transdermal delivery system can be occlusive, semi-occlusive or non-occlusive, and can be adhesive or non-adhesive.

The transdermal formulation comprising active agents can be incorporated within the patch and patch can be applied topically to the skin surface. The patch can be left on the subject for any suitable period of time.

In some embodiments, the transdermal patches provide for a constant rate of delivery of the active components of the transdermal patch over a predetermined time period. In some embodiments, the predetermined time period is 4 hours, 8 hours, 12 hours, 24 hours, 48 hours, 72 hours, 96 hours, 120 hours, 144 hours, 7 days, 8 to 13 days, two weeks, or 15 days.

In yet further embodiments, the transdermal patches described herein provide a steady absorption rate of the active components of the transdermal patches by the patient over a predetermined time. In some embodiments, the predetermined time period is 4 hours, 8 hours, 12 hours, 15 hours, 24 hours, 48 hours, 72 hours, 96 hours, 120 hours, 144 hours, 7 days, 8 to 13 days, two weeks, or 15 days. In yet further embodiments, the transdermal patches described herein provide a constant blood serum level of the active components of the transdermal patches in a patient over a predetermined time. In some embodiments, the predetermined time period is 4 hours, 8 hours, 12 hours, 24 hours, 48 hours, 72 hours, 96 hours, 120 hours, 144 hours, 7 days, 8 to 13 days, two weeks, or 15 days.

In yet further embodiments, the transdermal patches described herein provide a plasma concentration of the active components of the transdermal patches in a therapeutic range in a patient over a predetermined time. In some embodiments, the predetermined time period is 4 hours, 8 hours, 12 hours, 24 hours, 48 hours, 72 hours, 96 hours, 120 hours, 144 hours, 7 days, 8 to 13 days, two weeks, or 15 days.

In yet further embodiments, the transdermal patches described herein allow for reduced variability in dosage of active components in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a plasma concentration of the active components of the transdermal patches in a therapeutic range in a patient over a predetermined time. In exemplary embodiments as disclosed herein, the transdermal patch provides a blood serum level of active agent selected from without any limitation, for example, about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL and ranges thereof. In one aspect, transdermal patch provides a blood serum level of active agent in the range of 0.01 ng/mL-400 ng/mL. In another aspect, transdermal patch provides a blood serum level of active agent in the range of 0.01 ng/mL-100 ng/mL.

The topical formulation stated in the art which include, for example without any limitation, semisolids such as ointment, cream, emulsion, micro emulsion, nano emulsion, paste, balms, gels, lotions, mousses, film forming semisolid formulations such as but not limited to film forming gels, emulsions, microemulsion etc. Liquids such as solutions, suspensions, micro suspension, nano suspension, dispersions, nano dispersion etc. Sprays, aerosols, magma, film forming spray, film forming liquid, etc. The topical formulation can be topically applied to the skin surface for transdermal delivery of active agents such as THC and/or CBD and/or nabilone, and derivatives of these compounds.

The transdermal formulation and/or topical formulation of some embodiments of the present disclosure may include carriers or ingredients in effective amount either alone or in combinations thereof without any limitation to the following carriers or ingredients such as solvents, gelling agents, polymers, pressure sensitive adhesive polymers, adhesive polymers, biodegradable polymers, penetration enhancers, emollients, skin irritation reducing agents, buffering agents, pH stabilizers, solubilizers, suspending agents, dispersing agents, stabilizers, plasticizers, tackifiers, surfactants, volatile chemicals, antioxidants, oxidants, chelating agents, complexing agents, diluents, excipients, material to prepare patch, material to prepare matrix patch, material to prepare reservoir patch etc. Active agents may be dissolved, suspended, dispersed or uniformly mixed in the above stated single carrier, mixture of carriers and combinations of carrier. Any combination of two or more drugs may be dissolved, suspended, dispersed or uniformly mixed in the above stated single carrier, mixture of carriers and combinations of carrier.

The desired optimum transdermal and/or topical formulation as disclosed herein may comprise without any limitation to following carriers as stated from example 1 to example 12 either alone or in combinations thereof.

### Indications

According to certain embodiments, transdermal compositions described herein are for the treatment and/or prevention and/or management of symptoms and/or control of various indications in patients, as disclosed herein.

Multiple Sclerosis: According to certain embodiments, transdermal compositions described herein are for the treatment and/or prevention and/or control of multiple sclerosis, multiple sclerosis-related muscle spasms, and pain and/or spasticity in multiple sclerosis.
Certain unwanted side effects caused by current medications are reduced or eliminated when treating patients with the compounds disclosed herein. One major side effect of the currently used medications containing THC (or its analogs), such as a US-listed drug dronabinol (Marinol) (synthetic THC) or Sativex, is the debilitating psychotropic effect at higher therapeutic doses. The higher doses are often necessary for resistant patients to manage pain, depression, or motoric disfunctions associated with: neuropathic pain, such as allodynia in Complex Regional Pain Syndrome (CRPS), MS spasticity, Spinal Cord Injury (SCI)-related spasticity, Hereditary Spastic Paraplegia (HSP)-related spasticity, Spastic Diplegia Cerebral Palsy (SDCP)-related spasticity, Spinocerebellar Ataxias (SA)-related spasticity, Huntington's Disease (HD) chorea, Post Traumatic Stress Disorder (PTSD) depression, Tuberous Sclerosis (TS) tumors and inflammation, and others.
Neurodegenerative diseases, such as MS, are characterized by changes in normal neuronal functioning, leading, in most cases, to neuronal death. Studies in animals and humans demonstrate that changes also occur in the endocannabinoid. More specifically, multiple studies indicate that control of spasticity in MS is mediated by CB1, and not CB2 cannabinoid receptors, but the role of CB2 receptor cannot be entirely excluded.

There are multiple studies demonstrating spasticity inhibition of THC—the main psychotropic constituent of the Cannabis sativa plant (Petro & Ellenberger, Treatment of human spasticity with delta 9-tetrahydrocannabinol, 1981) (Ungerleider, Andyrsiak, Fairbanks, Ellison, & Myers, Delta-9-THC in the treatment of spasticity associated with multiple sclerosis, 1987). Also, a synthetic THC drug Marinol (United Pharmaceuticals) and THC derived Cesamet (Valeant Pharmaceuticals International) were tested and both demonstrated positive results in reducing spasticity. This is not surprising since THC is a CB1 and CB2 receptor partial agonist. Yet, CB1 receptor also mediates the THC-induced psychoactivity. Consequently, there is a mounting evidence of the unwanted psychotropic effects, especially with synthetic THC and natural THC at higher doses, such as intoxication, sedation, memory impairment, dysphoria (and adverse behavioral changes.

It is rather difficult to dissociate the therapeutic effects from the adverse effects of cannabinoids when using Cannabis as a medicine. It is, however, hypothesized that this aspect can be mitigated by administering a Cannabis extract that contains other cannabinoids in addition to THC. Cannabidiol—an antagonist of CB1 and CB2 receptors is believed to be modulating the THC psychotropic effect. However, the effective concentrations and ratios, pharmacodynamics, presence or absence of other substances that facilitate this complex interaction was not understood until now.

It is plausible that CBD functions as a negative allosteric modulator by binding to a site on the CB1 receptor distinct from the THC binding site. From this secondary site, the inventors conclude, CBD is able to change the shape of the receptor in a way that there is less THC binding and less activation of the CB1 receptor. CBD was shown to inhibit uptake and metabolism of anandamide. It is also suggested that at low concentrations CBD acts as an inverse agonist, binding to the same receptors that other agonists do but causing a different physiological effect. Reduced THC potency was seen at CBD concentrations as low as 100 nM that is well achievable in a human.

Further, in line with classical pharmacology, the responses THC elicits appear to be strongly influenced both by the expression level and signaling efficiency of cannabinoid receptors and by ongoing endogenous cannabinoid release.

CBD also inhibits the conversion of THC into its particularly psychoactive metabolites 11-hydroxy-THC, reducing psychoactive effects, yet, it works synergistically with THC on control of pain and spasticity. It is further suspected that CBD delays the absorption of THC, and consequently peak serum concentrations that are associated with the occurrence of unwanted side effects are avoided. It has been further observed that Cannabis strains, containing CBD levels equal or higher than THC, have positive effects on muscle spasticity. These positive effects of Cannabis on spasticity and pain and its safety have also been emphasized by the American Academy of Neurology (Koppel, et al., 2014). Moreover, CBD is presumed to have antipsychotic, anxiolytic, and anticonvulsant effects, and experimental evidence suggests that THC, as well as CBD, exhibit anti-inflammatory, neuroprotective, and immunomodulatory properties, pointing to additive or potentiating effects of the combination of the two.

Considering the known deficiencies of comparable commercial and experimental drugs, disclosed herein are new formulations that are more effective clinically for treating pain, inflammation, mood, spasticity, and motoric symptoms associated with psychiatric, autoimmune and neurological diseases and disorders, such as, but not limited to neuropathic, acute or chronic pain such as in CPRS, cancer or trauma; Ataxias (cerebellar, sensory, vestibular), such as tremor or vertigo; MS, HSP, SCI, SDCP, SA spasticity and pain, HD chorea; PD rigidity; PTSD depression; migraine; TS tumors and inflammation—all these symptoms are responsive to specific modulations of the endocannabinoid receptors and/or the neuroprotective/antioxidant qualities of specific cannabinoids.

Though, the clinical effect of combination therapy has been confirmed in studies and commercially with Sativex (THC/CBD combination where THC>CBD), it is apparent that the total dose of THC delivered in Sativex is not sufficient to saturate the receptors, not making full use of the medication's therapeutic potential. The literature on Sativex shows a large variance in daily doses. Sativex is administered as buccal spray, from 5 sprays per day (Johnson, Lossignol, Burnell-Nugent, & Fallon, 2013) to a maximum of 14 sprays (THC 2.7 mg: CBD 2.5 mg per spray) (Bayer HealthCare press release and Medical Update Memo of Multiple Sclerosis Society of Canada, 2005). At these high doses, not surprisingly, the unwanted psychotropic effects of THC become dominant, limiting the drug's utility.

To achieve the desired efficacy attainable at high THC doses, but overcoming the negative psychotropic effect, the inventor employs a balanced THC/CBD formula that ranges in some embodiments 0.5 to 1 (THC) and 1.5 to 2 (CBD) ratios, and in other embodiments 1 to 1.8 (THC) and 2 to 2.5 (CBD) ratios (from approximately 52% to 83% of CBD, where THC and CBD is 100%), whereas CBD in the ratios significantly higher than found in Sativex (relative to THC concentrations), would limit the THC psychotropic effect. In addition, in one embodiment, the formulation contains up to 50% of the total formulation other phytochemicals and impurities coextracted with THC and/or CBD from the Cannabis plant. And in another embodiment, THC and CBD represent at least 97% of the total formulation and the remaining 3% or less are impurities.

Considering the safety aspects associated with higher THC and CBD doses, the inventor believes, and it was experimentally determined in humans and animals, that aforesaid formulations and dosing is appropriate for patients, which is in addition corroborated by the following experiments with higher THC ratios and, in one case, a 200 mg high CBD dose: 200 mg CBD per day or 10 mg THCV per day or 10 mg CBD and 10 mg THCV per day or 200 mg CBD and 10 mg THCV per day were all well tolerated.

In one embodiment of the disclosure, the treatment of a patient with an autoimmune disease Rheumatoid arthritis involves giving to a patient every 12 hours by oral administration one ARCO® Chemie 60 Oval soft-gel capsule of the compound containing a mixture of 5 mg delta-9 and delta-8-tetrahydrocannabinol (THC) and cannabidiol (CBD) in the ratio of approximately 0.7 (THC) and 2 (CBD) by mass, and such mixture contains less than 5 mg of other cannabinoids co-extracted with THC and CBD from the Cannabis indica plant and not completely removed from the extract, and in one embodiment, one or more non-cannabinoid components, such as sesame oil and a mixture of parahydroxybenzoates, where such capsule is a time-released capsule designed to release said compound in the small intestine; and in another embodiment, in the stomach. The aforesaid compound and method have statistically meaningfully reduced perception of pain on the Rheumatoid Arthritis Pain Scale (RAPS) in subjects suffering from pain associated with Rheumatoid Arthritis inflammation after being administered for 5 days, having minimal to nonexistent debilitating psychotropic effect. RAPS measures physiological, sensory-discriminative, and cognitive components.

Neurological Disorders: As used herein, “central nervous system” (CNS) refers to the brain and spinal cord and associated tissues. As used herein, “neurological disorders and diseases of the CNS” refers to brain diseases and conditions that comprise ischemia, i.e., cerebral ischemia, ischemia, stroke, neurodegeneration, neurological complications arising from such as Alzheimer's disease, Parkinson's disease, Wilson's disease, Lewy body dementia, multiple sclerosis, seizure disorders, cerebellar ataxia, progressive supranuclear palsy, amyotrophic lateral sclerosis, autism, affective disorders, anxiety disorders, metabolic disorders that affect the CNS, and/or schizophrenia; cell damage; nerve damage from cerebrovascular disorders such as stroke in the brain or spinal cord, from CNS infections including meningitis and HIV, from tumors of the brain and spinal cord, prion diseases, and CNS disorders resulting from ordinary aging (e.g., anosmia), head and/or brain injury, or spinal cord injury and any other medical diseases and conditions mentioned herein with neurological cell loss, damage and/or degeneration.

Other conditions are also contemplated herein, that include, but are not limited to: gastrointestinal, metabolic, neurological, circulatory, soft tissue, musculoskeletal, chronic or acute pain, nausea, decreased appetite, skin disorders, sexual dysfunction, glaucoma, AIDS wasting, neuropathic pain, treatment of spasticity associated with multiple sclerosis, fibromyalgia, chemotherapy-induced nausea, allergies, inflammation, infection, epilepsy, depression, migraine, bipolar disorders, anxiety disorder, dependency and withdrawal. In addition, the methods of the disclosure may be used to alleviate, or relief symptoms or side effects associated with anti-retroviral therapy, chemotherapy and radiation therapy. Certain diseases and disorders are briefly outlined below, and the possible mechanisms of cannabinoid action are exemplified with treatment of certain autoimmune neurodegenerative diseases.

Neurodegenerative diseases, such as MS, are a group of disorders characterized by changes in normal neuronal functioning, leading, in most cases, to neuronal death. Most of these diseases are associated, especially in late stages, with severe neuronal loss. With an ever-increasing ageing population, progressively more individuals are affected by neurodegenerative diseases. According to the National Institute of Neurological Disorders and Stroke, there are more than 600 different types of neurological disorders.

Neural degeneration, or neurodegeneration, can be described as the progressive damage or death of neurons. Neurons are nerve cells in the brain whose primary function is to assist in the memory process. The damage or death of neurons leads to a gradual deterioration of the functions controlled by the affected part of the nervous system. Neural degeneration often occurs because of oxidative stress. Oxidative stress occurs to the cells when the effects of pro-oxidants (such as free radicals, reactive oxygen and reactive nitrogen species) exceed the ability of anti-oxidants to neutralize them. When levels of free radicals or other pro-oxidants increase to such an extent, they can cause damage to cell membranes which in turn may result in cell death or damage to genetic material.

Some of the most common types of neurological disorders include Alzheimer's disease, Parkinson's disease and MS. The process of neural degeneration is often the result of glutamate excitotoxicity. Glutamate is a signaling chemical and under normal conditions the concentration of glutamate in a cell tends to be quite low. Glutamate is required at these low concentrations for crucial brain functions such as memory and learning. When glutamate concentrations increase, the process of neural degeneration begins.

When the brain is deprived of oxygen either due to a disease, such as a neurogenerative disease, a trauma, such as a closed head injury or due to an ischemic event such as a stroke, an abnormal build-up of glutamate occurs. Neural degeneration takes place when glutamate attaches to receptor proteins on a cells surface. Neural degeneration continues from the destructive effects of oxidative radicals caused by the glutamate flood. The radicals cause disruption of essential reactions in the neurons and this leads to degeneration or death of the cell.

Neuroprotective agents that can block the NMDA receptor are useful as they are able to block the reaction caused by glutamate and therefore prevent neural degeneration. Some neuroprotective agents, which block the NMDA receptor, have been studied in clinical trials in stroke patients, such as Dextrorphan, Selfotel, and Cerestat.

The motor neuron diseases (MNDs) are a group of progressive neurological disorders that destroy motor neurons, the cells that control essential voluntary muscle activity such as speaking, walking, breathing, and swallowing. Normally, messages from nerve cells in the brain (called upper motor neurons) are transmitted to nerve cells in the brain stem and spinal cord (called lower motor neurons) and from them to muscles. Upper motor neurons direct the lower motor neurons to produce movements such as walking or chewing. Lower motor neurons control movement in the arms, legs, chest, face, throat, and tongue.

When there are disruptions in the signals between the lowest motor neurons and the muscle, the muscles do not work properly; the muscles gradually weaken and may begin wasting away and develop uncontrollable twitching (fasciculations). After there are disruptions in the signals between the upper motor neurons and the lower motor neurons, the limb muscles develop stiffness (spasticity), movements become slow and effortful, and tendon reflexes such as knee and ankle jerks become overactive. Over time, the ability to control voluntary movement can be lost. Some MNDs are inherited, but the causes of most MNDs are not known. In sporadic or non-inherited MNDs, environmental, toxic, viral, or genetic factors may be implicated. Amyotrophic lateral sclerosis (ALS), progressive bulbar palsy, pseudobulbar palsy, primary lateral sclerosis (PLS), progressive muscular atrophy, spinal muscular atrophy (SMA), post-polio syndrome (PPS) are among the frequently encountered MNDs.

Amyotrophic lateral sclerosis is an MND that affects over 350,000 of the world's population and kills over 100,000 every year. Early symptoms associated with ALS include tripping, dropping things, slurred or “thick” speech, and muscle cramping, stiffening, weakening, and twitching (fasciculation). In bulbar ALS, the muscles for speaking, swallowing or breathing are affected. In addition to muscle loss atrophy, the following signs of lower and upper motor neuron degeneration are often associated with ALS lower motor neuron degeneration: muscle weakness and atrophy, involuntary contraction of muscle fibers, muscle cramps, weakened reflexes, flaccidity (decreased muscle tone), difficulty swallowing, disordered articulation, shortness of breath at rest, etc. Upper motor neuron degeneration: muscle stiffness or rigidity, emotional lability (decreased ability to control emotions). in an embodiment, the present disclosure provides a method of treating/preventing/ameliorating a disease, disorder or condition selected from a group comprising of pain, cancer, pain, cancer, glaucoma, HIV, neurodegeneration, Alzheimer's disease, Parkinson's disease, multiple sclerosis, psychosis, anorexia/cachexia, nausea, hepatitis B, depression, anxiety, paraplegia, rheumatoid arthritis, sleeplessness, epilepsies, neurological movement disorders, Inflammation, Crohn's diseases, ulcerative colitis, arthritis, neurodermatitis cognitive impairment, dementia, diabetes mellitus and hypertension by administering a therapeutically effective amount of the composition to a subject in need thereof.

Chronic Pain: One embodiment of the disclosure is the pharmaceutical composition for use in the treatment of chronic pain according to the disclosure, wherein the composition contains active agent as disclosed herein, and wherein the composition is administered every other day, daily, twice daily, three times daily or four times daily or six times daily for a period of at least one day, at least one week, anytime between one day to one week, anytime between one week to one year, at least one year, or longer. One embodiment of the disclosure is the pharmaceutical composition for use in the treatment of chronic pain according to the disclosure, wherein the composition is administered every other day, daily, twice daily, three times daily or four times daily for a period of at least one day, at least one week, anytime between one week to one year, at least one year, or longer. This way, a continuous decrease of (peripheral) neuropathic pain, inflammatory pain, musculoskeletal pain, pain due to muscle spasms, and/or other chronic pain states is achieved upon administering the pharmaceutical composition of the disclosure to a patient suffering from chronic pain.

In one embodiment, the pharmaceutical composition for use in the treatment of chronic pain according to the disclosure is a pharmaceutical transdermal composition wherein the use is the transdermal use in the treatment of chronic pain according to the disclosure.

In one embodiment, the pharmaceutical composition for use in the treatment of chronic pain according to the disclosure is a pharmaceutical topical composition wherein the use is the topical use in the treatment of chronic pain according to the disclosure. Here, topical composition will be topically applied to intact skin area experiencing pain in the treatment of chronic pain.

In one embodiment, the pharmaceutical composition for use in the treatment of chronic pain according to the disclosure is a pharmaceutical transdermal composition wherein the use is the transdermal use on intact skin of the treated person in the treatment of chronic pain according to the disclosure.

In one embodiment, the pharmaceutical composition for use in the treatment of chronic pain according to the disclosure, is a pharmaceutical transdermal composition wherein the use is the transdermal use on healthy intact skin of the treated person in the treatment of chronic pain according to the disclosure. Here, intact skin and healthy intact skin have their common scientific meaning and here refer to non-injured skin free of e.g., ulcers, wounds, lesions, cuts, and refer to skin comprising a closed outer layer of epidermis.

One embodiment of the disclosure is a pharmaceutical composition according to the disclosure or provided by the method of the disclosure, for use in the treatment of chronic pain according to the disclosure, wherein the chronic pain is neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, or combinations thereof.

In one embodiment, the pharmaceutical composition for use in the treatment of chronic pain according to the disclosure is the pharmaceutical composition, wherein the chronic pain is peripheral neuropathic pain.

One embodiment of the disclosure is a pharmaceutical composition according to the disclosure or provided by the method of the disclosure, for use in the treatment of chronic pain according to the disclosure, wherein the chronic pain is neuropathic pain selected from peripheral neuropathy caused by diabetes type 1 or 2, or induced by a noxious substance such as alcohol, due to vitamin B1, B6 and/or B12 deficiency, hypervitaminosis B6, hypothyroidism, chemotherapeutic compound such as paclitaxel or a taxane derivative, vincristine or a vinca alkaloid, cisplatin or a platinum derivate, drug-induced neuropathy, a compound for the treatment of infectious disease such as streptomycin, didanosine or zalcitabine, a chemically toxic compound, trigeminal neuralgia, post-herpetic neuralgia, intercostal neuralgia, entrapment neuropathy such as carpal tunnel syndrome, tarsal tunnel syndrome, abdominal cutaneous nerve entrapment syndrome, sciatic pain chronic idiopathic sensory neuropathy, small fiber neuropathy, hereditary motor and sensory neuropathies, chronic inflammatory demyelinating polyneuropathy, infectious disease conditions such as post-polio syndrome, AIDS or HIV-associated, Lyme-associated, Sjogren-associated, lymphomatous neuropathy, myelomatous neuropathy, carcinomatous neuropathy, acute pan autonomic neuropathy, vasculitic/ischaemic neuropathy and a mono- and polyneuropathy, complex regional pain syndrome type I and II (reflex sympathetic dystrophy), central neuropathic pain such as thalamic neuropathy, spinal cord injury neuropathy, post stroke pain, multiple sclerosis neuropathy, syringomyelia, a spinal cord tumor, phantom limb pain, restless genital syndrome with pain, post-surgical scar pain including scar pain after cardiac surgery and mastectomy.

One embodiment of the disclosure is a pharmaceutical composition according to the disclosure or provided by the method of the disclosure, for use in the treatment of chronic pain according to the disclosure, wherein the dosing frequency of the pharmaceutical composition is between once every other day and eight times daily, preferably six, five, four, three, two or one times daily.

One embodiment of the disclosure is the pharmaceutical composition for use in the treatment of chronic pain according to the disclosure, wherein the pharmaceutical composition is administered during a period of at least one day, preferably at least one week, more preferably at least one month, most preferably at least one year, preferably the pharmaceutical composition is administered for one to ten years, more preferably the pharmaceutical composition is administered chronically. It is to be understood that it is part of the disclosure that the pharmaceutical composition for use in the treatment of chronic pain according to the disclosure is administered to patients suffering from chronic pain for the rest of their lifespan. This way, the chronic pain is at least less intense and preferably patients are relieved from the chronic pain to a large extent or even completely.

Chemotherapy Induced Nausea and Vomiting: The disclosure relates to the field of transdermal and/or topical delivery system of pharmaceutical composition or formulation comprising therapeutically effective amount of Cannabis and its derivatives, and other active agents, for example, to treat LGS, Dravet Syndrome, TSC, chronic pain, neuropathic pain, nausea and vomiting associated with chemotherapy which may include Cannabis and its derivatives or its pharmaceutically acceptable salts which can be anhydrous and/or hydrous, and/or amorphous, and/or crystalline, and/or co-crystals either alone or in combinations thereof. More preferably transdermal formulation may include, for example, cannabidiol in the following forms: free base or salt or co-crystal or crystalline or amorphous or isomers or prodrug or derivatives or metabolites either alone or in combination of thereof. Nausea and vomiting is a human reflex against the absorption of toxins which include many chemotherapeutic agents. Chemotherapy induced nausea and vomiting (CINV) is not entirely understood, however, it is thought to have many contributing pathways which serve as potential targets for therapy. A complex condition involving multiple mechanisms, CINV has been classified into five categories. Acute CINV occurs in the first 24 hours after chemotherapy and is primarily due to the activation of the serotonin receptors in the gastrointestinal tract. Delayed CINV is nausea and/or vomiting that develops more than 24 hours after chemotherapy administration. Breakthrough CINV is vomiting and/or nausea that occurs within five days of chemotherapy administration after the use of guideline directed prophylactic antiemetic agents. Refractory CINV occurs after chemotherapy in subsequent chemotherapy cycles after guideline directed prophylactic agents have failed in earlier cycles. Anticipatory CINV may develop in patients if they experience CINV after chemotherapy administration despite prophylactic antiemetics.

The incidence and onset of CINV varies among patients, however, studies have led to the classification of chemotherapeutic agents based on their likelihood of inducing CINV. Chemotherapeutic agents can be classified as highly emetogenic (HEC) or moderately emetogenic (MEC). Highly emetic agents have a greater than 90% risk of emesis while moderately emetic agents have a 30% to 90% risk of emesis. Low risk and minimal risk emetogenic chemotherapeutics have a 10% to 30% chance and a less than 10% chance of causing CINV, respectively. Some chemotherapy regimens require no prophylaxis, whereas others require prophylaxis with medications from multiple classes. Unfortunately, some subjects still experience CINV despite administration of appropriate prophylaxis.

Extensive studies of the pathophysiology of CINV have led to the development of three classes of antiemetics commonly used for treatment and prophylactic treatment of CINV: 5-HT3 receptor antagonists, glucocorticoids, and NK-1 receptor antagonists.

Some chemotherapeutic agents cause the release of large amounts of serotonin by enterochromaffin cells in the small intestine stimulating vagal afferent nerves via the 5-HT3 receptors, thereby initiating the vomiting reflex. The development of specific 5-HT3 receptor antagonists has improved the treatment of the nausea and vomiting that often accompanies chemotherapeutic regimens. Therapeutically effective 5-HT3 receptor antagonists include, for example, granisetron, ondansetron, dolasetron, tropisetron, and palonosetron. 5-HT3 receptor antagonists have also been proven to be effective in treating post-operative nausea and vomiting (PONY). Another mediator of vomiting pathways is substance P, a tachykinin family neuropeptide which binds to and activates the neurokinin-1 (NK-1) receptor. Accordingly, NK-1 receptor antagonists have been developed which are therapeutically effective in treating CINV and include aprepitant, fosaprepitant, netupitant, and rolapitant. NK-1 receptor antagonists are often administered in combination with 5-HT3 receptor antagonists to treat CINV.

More recently, the antipsychotic agent olanzapine has also been shown to have therapeutic value in the treatment of CINV (Brafford, 2014, J Adv Pract Oncol, 5:24-29). Olanzapine targets and blocks many different receptors such as dopaminergic, serotonergic, adrenergic, histaminergic, and muscarinic receptors. Thus, olanzapine has the advantage of targeting multiple key receptors with a single medication and has become an increasingly attractive agent for development of its use in treating CINV.

Current treatment and prophylactic treatment of CINV often involves combinations of the various antiemetics. Specifically, 5-HT3 receptor antagonists are often used in conjunction with glucocorticoid steroids and NK1 receptor antagonists. For example, acute CINV is often treated with a combination of a 5-HT3 receptor antagonist, dexamethasone and an NK-1 receptor antagonist. NK-1 receptor antagonists are also often used for treating delayed CINV. Despite the therapeutic efficacy of these regimes, there still exists a need for optimizing antiemetic therapies involving the use of olanzapine to address issues such as breakthrough CINV, reduction of side effects of currently recommended regimes, and convenience for patients undergoing chemotherapy.

Of particular interest is a composition for the extended release of olanzapine and a 5-HT3 receptor antagonist from a polymeric formulation which provides therapeutic efficacy over a period of at least 5 days. In a particular embodiment, the extended release composition comprising a polyorthoester, olanzapine and granisetron. The compositions disclosed herein are useful for the treatment and/or prophylactic treatment of CINV in subjects undergoing chemotherapy, including acute, delayed, breakthrough and refractory CINV. However, it is understood that the compositions may also be useful in treating or prophylactically treating PONY in subjects in need thereof. In some embodiments, the nausea associated symptoms are selected from the group consisting of: motion sickness, chemotherapy induced nausea vomiting (CINV), pregnancy (morning sickness), anxiety induced irritable bowel syndrome, Crohn's Disease, medication induced dizziness, vertigo, stress, migraines, general anxiety, and depression.

Epilepsy: According to certain embodiments, transdermal compositions described herein are for the treatment and/or prevention and/or control of seizure disorder in a patient, wherein the seizure disorder disorders include, for example, complex partial seizures, simple partial seizures, partial seizures with secondary generalization, generalized seizures (including absence, grand mal (tonic clonic), status epilepticus, tonic, atonic, myoclonic), neonatal and infantile spasms, drug induced seizures, trauma-induced seizures, and febrile seizures, and additional specific epilepsy syndromes such as juvenile myoclonic epilepsy, Lennox-Gastaut, Dravet syndrome, mesial temporal lobe epilepsy, nocturnal frontal lobe epilepsy, progressive epilepsy with mental retardation, and progressive myoclonic epilepsy, as well as seizures associated with CNS mass lesions.

Epilepsy is a brain disorder characterized by repeated seizures over time. Types of epilepsy can include, but are not limited to generalized epilepsy, e.g., childhood absence epilepsy, juvenile myoclonic epilepsy, epilepsy with grand-mal seizures on awakening, West syndrome, Lennox-Gastaut syndrome, Dravet syndrome, partial epilepsy, e.g., temporal lobe epilepsy, frontal lobe epilepsy, benign focal epilepsy of childhood.

Status epilepticus (SE) can include, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges. Convulsive status epilepticus is characterized by the presence of convulsive status epileptic seizures, and can include early status epilepticus, established status epilepticus, refractory status epilepticus, super-refractory status epilepticus. Early status epilepticus is treated with a first line therapy. Established status epilepticus is characterized by status epileptic seizures which persist despite treatment with a first line therapy, and a second line therapy is administered.

Refractory status epilepticus is characterized by status epileptic seizures which persist despite treatment with a first line and a second line therapy, and a general anesthetic is generally administered. Super refractory status epilepticus is characterized by status epileptic seizures which persist despite treatment with a first line therapy, a second line therapy, and a general anesthetic for 24 hours or more. Non-convulsive status epilepticus can include, e.g., focal non-convulsive status epilepticus, e.g., complex partial non-convulsive status epilepticus, simple partial non-convulsive status epilepticus, subtle non-convulsive status epilepticus; generalized non-convulsive status epilepticus, e.g., late onset absence non-convulsive status epilepticus, atypical absence non-convulsive status epilepticus, or typical absence non-convulsive status epilepticus.

Compositions described herein can also be administered as a prophylactic to a subject having a CNS disorder e.g., a traumatic brain injury, status epilepticus, e.g., convulsive status epilepticus, e.g., early status epilepticus, established status epilepticus, refractory status epilepticus, super-refractory status epilepticus; non-convulsive status epilepticus, e.g., generalized status epilepticus, complex partial status epilepticus; generalized periodic epileptiform discharges; and periodic lateralized epileptiform discharges; prior to the onset of a seizure.

Seizure: A seizure is the physical findings or changes in behavior that occur after an episode of abnormal electrical activity in the brain. The term “seizure” is often used interchangeably with “convulsion.” Convulsions are when a person's body shakes rapidly and uncontrollably. During convulsions, the person's muscles contract and relax repeatedly.

Based on the type of behavior and brain activity, seizures are divided into two broad categories: generalized and partial (also called local or focal). Classifying the type of seizure helps doctors diagnose whether or not a patient has epilepsy.

Generalized seizures are produced by electrical impulses from throughout the entire brain, whereas partial seizures are produced (at least initially) by electrical impulses in a relatively small part of the brain. The part of the brain generating the seizures is sometimes called the focus.

There are six types of generalized seizures. The most common and dramatic, and therefore the most well-known, is the generalized convulsion, also called the grand-mal seizure. In this type of seizure, the patient loses consciousness and usually collapses. The loss of consciousness is followed by generalized body stiffening (called the “tonic” phase of the seizure) for 30 to 60 seconds, then by violent jerking (the “clonic” phase) for 30 to 60 seconds, after which the patient goes into a deep sleep (the “postictal” or after-seizure phase). During grand-mal seizures, injuries and accidents may occur, such as tongue biting and urinary incontinence.

Absence seizures cause a short loss of consciousness (just a few seconds) with few or no symptoms. The patient, most often a child, typically interrupts an activity and stares blankly. These seizures begin and end abruptly and may occur several times a day. Patients are usually not aware that they are having a seizure, except that they may be aware of “losing time.”

Myoclonic seizures consist of sporadic jerks, usually on both sides of the body. Patients sometimes describe the jerks as brief electrical shocks. When violent, these seizures may result in dropping or involuntarily throwing objects.

Clonic seizures are repetitive, rhythmic jerks that involve both sides of the body at the same time; Tonic seizures are characterized by stiffening of the muscles; Atonic seizures consist of a sudden and general loss of muscle tone, particularly in the arms and legs, which often results in a fall. Seizures described herein can include epileptic seizures; acute repetitive seizures; cluster seizures; continuous seizures; unremitting seizures; prolonged seizures; recurrent seizures; status epilepticus seizures, e.g., refractory convulsive status epilepticus, non-convulsive status epilepticus seizures; refractory seizures; myoclonic seizures; tonic seizures; tonic-clonic seizures; simple partial seizures; complex partial seizures; secondarily generalized seizures; atypical absence seizures; absence seizures; atonic seizures; benign Rolandic seizures; febrile seizures; emotional seizures; focal seizures; gelastic seizures; generalized onset seizures; infantile spasms; Jacksonian seizures; massive bilateral myoclonus seizures; multifocal seizures; neonatal onset seizures; nocturnal seizures; occipital lobe seizures; post traumatic seizures; subtle seizures; Sylvan seizures; visual reflex seizures; or withdrawal seizures.

The invention will be illustrated in more detail with reference to the following Examples, but it should be understood that the present invention is not deemed to be limited thereto.

### EXAMPLES

### Example 1

The transdermal formulation and/or topical formulation of the disclosure may comprise solvents known to those skilled in the art either alone or in combinations thereof without any limitation to following like alcohol C₁-C₂₀ such as but not limited to (methanol, ethanol, isopropyl alcohol, butanol, propanol etc.), polyhydric alcohols, glycols such as but not limited to (propylene glycol, polyethylene glycol, dipropylene glycol, hexylene glycol, butyene glycol, glycerine etc.), derivative of glycols, pyrrolidone such as but not limited to (N methyl 2-pyrrolidone, 2-pyrrolidone etc.), sulfoxides such as but not limited to (dimethyl sulfoxide, decymethylsulfoxide etc), dimethylisosorbide, mineral oils, vegetable oils, sesame oil, water, polar solvents, semi polar solvents, non polar solvents, volatile chemicals which can be used to make matrix patch such as but not limited to (ethanol, propanol, ethyl acetate, acetone, methanol, dichloromethane, chloroform, toluene, IPA, hexane, etc), pentane, dimethylformamide, butane, lipids acids such as but not limited to acetic acid, lactic acid, levulinic acid, bases and others. More preferably in the range of 0.01%-95% w/w or w/v. In exemplary embodiments, formulations of the disclosure may comprise solvents at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 76%, and about 80%, and about 95% of the formulation. In exemplary embodiments, formulations of the disclosure may comprise solvents at a concentration of about 1 to 20%, of about 5% to 25%, about 10% to about 20%, or about 15% to about 18%, about 30% to about 70%, about 35% to about 65%, about 66%, and about 40% to about 64% w/w. In exemplary formulations of the disclosure, the solvents will represent approximately 1 wt % to 75 wt %, preferably 2 wt % to 30 wt %, more preferably 5 wt. % to 20 wt. % of the formulation.

### Example 2

The transdermal formulation and/or topical formulation of the disclosure may comprise gelling agents and/or thickening and/or suspending agents and/or polymers and/or adhesive polymers and/or pressure sensitive adhesive polymers, biodegradable polymers known to those skilled in the art either alone or in combinations thereof without any limitation to following like natural polymers, polysaccharides and its derivatives such as but not limited to (agar, alginic acid and derivatives, cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose, hydroxylpropylmethyl cellulose etc.), synthetic polymers and its derivatives such as without any limitation to carboxyvinyl polymers or carbomers (carbopol 940, carbopol 934, carbopol 971p NF), polyethylene, and its copolymers etc, clays such as but not limited to (silicates, bentonite), silicon dioxide, polyvinyl alcohol, acrylic polymers (eudragit), acrylic acid esters, polyacrylate copolymers, polyacrylamide, polyvinyl pyrrolidone homopolymer and polyvinyl pyrrolidone copolymers such as but not limited to (PVP, Kollidon 30, poloxamer), isobutylene, ethyl vinyl acetate copolymers, natural rubber, synthetic rubber, pressure sensitive adhesives such as silicone polymers such as but not limited to (bio psa 4302, bio-psa 4202 etc.), acrylic pressure sensitive adhesives such as but not limited to (duro-tak 87-2156, duro-tak 387-2287, duro-tak 87-9301, duro-tak 387-2051 etc.), etc.), polyisobutylene such as but not limited to (polyisobutylene low molecular weight, plyisobutylene medium molecular weight, polyisobutylene 35000 mw, etc), acrylic copolymers, rubber based adhesives, hot melt adhesives, styrene-butadiene copolymers, bentonite, all water and/or organic solvent swellable polymers, etc. In exemplary embodiments, formulations of the disclosure may comprise gelling agents and/or thickening and/or suspending agents and/or polymers and/or adhesive polymers and/or pressure sensitive adhesive polymers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%, about 90%, about 95% of the formulation. In exemplary embodiments, formulations of the disclosure may comprise gelling agents and/or thickening and/or suspending agents and/or polymers and/or adhesive polymers and/or pressure sensitive adhesive polymers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the gelling agents and/or thickening and/or suspending agents and/or polymers and/or adhesive polymers and/or pressure sensitive adhesive polymers will represent approximately 1 wt % to 75 wt %, preferably 2 wt % to 30 wt %, more preferably 5 wt. % to 20 wt. % of the formulation, and more preferably in the range of 0.1%-95% w/w or w/v.

### Example 3

The transdermal formulation and/or topical formulation of the disclosure may comprise permeation enhancers known to those skilled in the art either alone or in combination thereof without any limitation to the following, such as sulfoxides, and similar chemicals such as but not limited to (dimethylsulfoxide, dimethylacetamide, dimethylformamide, decymethylsulfoxide, dimethylisosorbide etc), azone, pyrrolidones such as but not limited to (N-methyl-2-pyrrolidone, 2-pyrrolidon etc.), esters, fatty acid esters such as but not limited to (propylene glycol monolaurate, butyl ethanoate, ethyl ethanoate, isopropyl myristate, isopropyl palmitate, methyl ethanoate, decyloleate, glycerol monooleate, glycerol monolaurate, lauryl laurate, lauryl lactate, ethyl oleate, etc.), fatty acids such as but not limited to (capric acid, caprylic acid, lauric acid, oleic acid, myristic acid, linoleic acid, stearic acid, palmitic acid etc.), alcohols, fatty alcohols and glycols such as but not limited to (oleyl alcohol, nathanol, dodecanol, propylene glycol, glycerol etc.), ethers alcohol such as but not limited to (diethylene glycol monoethyl ether), urea, triglycerides such as but not limited to triacetin, polyoxyethylene fatty alcohol ethers, polyoxyethylene fatty acid esters, esters of fatty alcohols, essential oils, surfactant type enhancers such as but not limited to (brij, sodium lauryl sulfate, tween, polysorbate), terpene, terpenoids and all penetration or permeation enhancers referred in the book “Percutaneous Penetration Enhancers” (Eric W. Smith, Howard I. Maibach, 2005. November, CRC press). In exemplary embodiments, formulations of the disclosure may comprise permeation enhancers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise permeation enhancers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the permeation enhancers will represent approximately 1 wt % to 75 wt %, preferably 2 wt % to 30 wt %, more preferably 5 wt. % to 20 wt. % of the formulation. More preferably in the range of 0.01%-95% w/w or w/v.

### Example 4

The transdermal formulation and/or topical formulation of the disclosure may comprise plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tec Publishing). In exemplary embodiments, formulations of the disclosure may comprise plasticizers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation.

In exemplary embodiments, formulations of the disclosure may comprise plasticizers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the plasticizers will represent approximately 1 wt % to 75 wt %, preferably 2 wt % to 30 wt %, more preferably 5 wt. % to 20 wt. % of the formulation. More preferably in the range of 0.01% 5-95% w/w or w/v.

### Example 5

The transdermal formulation and/or topical formulation of the disclosure may comprise emollients, humectants, skin irritation reducing agents and similar compounds or chemicals known to those skilled in the art either alone or in combinations thereof without any limitation to following like petrolatum, lanolin, mineral oil, dimethicone, zinc oxide, glycerin, propylene glycol and others. More preferably in the range of 0.01%-95% w/w or w/v. In exemplary embodiments, formulations of the disclosure may comprise emollients, humectants, skin irritation reducing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise emollients, humectants, skin irritation reducing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the emollients, humectants, skin irritation reducing agents and similar compounds will represent approximately 1 wt % to 75 wt %, preferably 2 wt % to 30 wt %, more preferably 5 wt. % to 20 wt. % of the formulation; More preferably in the range of 0.01%-95% w/w or w/v.

### Example 6

The transdermal formulation and/or topical formulation of the disclosure may comprise solubilizers, surfactants, emulsifying agents, dispersing agents, diluents and similar compounds or chemicals known to those skilled in the art either alone or in combination thereof without any limitation to following like polysorbate such as but not limited to (polysorbate 20, polysorbate 40, polysorbate 60, polysorbate 80 etc.), span such as but not limited to (span 80, span 20 etc.), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl10 polyoxyl-6-glycerides, lauroyl polyoxyl-6-gylcerides, polyglyceryl-3-dioleate, diethylene glycol monoethyl ether, propylene glycol monolaurate type I, polyglyceryl-3-dioleate, caprylocaproyl polyoxyl-8 glycerides etc, cyclodextrins and others. Diluents known to those skilled in the art either alone or in combination thereof without any limitation to following like lactose, silicone dioxide, sucrose, mannitol, starch, fillers, etc. More preferably in the range of 0.01% 95% w/w or w/v. In exemplary embodiments, formulations of the disclosure may comprise solubilizers, surfactants, emulsifying agents, dispersing agents, diluents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solubilizers, surfactants, emulsifying agents, dispersing agents, diluents, and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solubilizers, surfactants, emulsifying agents, dispersing agents and similar compounds will represent approximately 1 wt % to 75 wt %, preferably 2 wt % to 30 wt %, more preferably 5 wt. % to 20 wt. % of the formulation, more preferably in the range of 0.01% 95% w/w or w/v.

### Example 7

Different techniques and ingredients can be used to increase the stability and/or solubility of the active agents in formulation such as without any limitation to coating, encapsulation, microencapsulation, nanoencapsulation, lyophilization, chelating agents, complexing agents, packaging of transdermal delivery system, moisture scavengers, oxygen scavengers, etc. Different stabilizers can be used to increase stability of active agents in formulation or transdermal drug delivery system.

### Example 8

The transdermal formulation and/or topical formulation of the disclosure may comprise auxiliary pH buffering agents and pH stabilizers and similar compounds known to those skilled in the art which helps to maintain the appropriate pH of formulation preferably in the range of 4.0-8.0 either alone or in combination thereof without any limitation to following such as phosphate buffer, acetate buffer, citrate buffer, etc., acids such as but not limited to (carboxylic acids, inorganic acids, sulfonic acids, vinylogous carboxylic acids and others), base such as but not limited to (sodium hydroxide, potassium hydroxide, ammonium hydroxide, triethylamine, sodium carbonate, sodium bicarbonate) etc. More preferably in the range of 0.01%-30% w/w or w/v. In exemplary embodiments, formulations of the disclosure may comprise pH buffering agents and pH stabilizers and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise pH buffering agents and pH stabilizers and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the pH buffering agents and pH stabilizers and similar compounds will represent approximately 1 wt % to 75 wt %, preferably 2 wt % to 30 wt %, more preferably 5 wt. % to 20 wt. % of the formulation. More preferably in the range of 0.01%-30% w/w or w/v.

### Example 9

The transdermal formulation and/or topical formulation of the disclosure may comprise antioxidants such as but not limited to (sodium metabisulfite, citric acid, ascorbic acid, BHA, BHT), oxidizing agents, stabilizers, discoloring agents, preservatives and similar compounds or chemicals known to those skilled in the art which helps to get a stable formulation can be used either alone or in combination thereof without any limitation. More preferably in the range of 0.01%-50% w/w or w/v. In exemplary embodiments, formulations of the disclosure may comprise antioxidants at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise antioxidants at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the antioxidants will represent approximately 1 wt % to 75 wt %, preferably 2 wt % to 30 wt %, more preferably 5 wt. % to 20 wt. % of the formulation, more preferably in the range of 0.01%-50% w/w or w/v.

### Example 10

The transdermal formulation and/or topical formulation of the disclosure may be formulated in forms such as in ointment base, cream base, gel base, known to those skilled in the art.

### Example 11

Materials to make the transdermal delivery system and/or topical delivery system of the disclosure in patch form known to those skilled in the art, for example, such as but not limited to reservoir patch, matrix patch, drug in adhesive matrix patch, transdermal films, film forming formulation, film forming spray, micro-dosing patch and may include, such as but are not limited to polymers, copolymers, derivatives, backing film, release membranes, release liners, etc. either alone or in combinations thereof. Pressure sensitive adhesives (such as but not limited to silicone polymers, rubber based adhesives, acrylic polymers, acrylic copolymers, polyisobutylene, acrylic acid-isooctyl acrylate copolymer, hot melt adhesives, polybutylene etc.), backing film (such as but not limited to ethylene vinyl acetate copolymers, vinyl acetate resins, polyurethane, polyvinyl chloride, metal foils, polyester, aluminized films, polyethylene, etc.), release membrane (such as but not limited to microporous polyethylene membrane, microporous polypropylene membrane, rate controlling ethylene vinyl acetate copolymer membrane etc.), release liners (such as but not limited to siliconized polyester films, fluoropolymer coated polyester film, polyester film, siliconized polyethylene terephthalate film, etc.) tapes, etc. The transdermal formulation and/or topical formulation and/or transdermal delivery system and/or topical delivery system of the disclosure may deliver at least therapeutic effective dose of active agent, such as for example, THC and/or CBD, and derivatives of these compounds, alone or in combinations thereof in human plasma required for treating and/or preventing and/or controlling chronic pain. Therapeutically effective active agent such as THC and/or CBD, and derivatives of these compounds dosages refers to the therapeutic concentration of in human plasma required for treating and/or preventing and/or controlling chronic pain. Furthermore, the precise therapeutic effective dose of such as THC and/or CBD, and derivatives of these compounds in the transdermal formulation or topical formulation or transdermal delivery system and/or topical delivery system can be determined by those skilled in the art based on factors such as but not limited to the patient's condition etc. The transdermal formulation or topical formulation or transdermal delivery system and/or topical delivery system will be available in different dosage strengths and patch sizes in order to achieve optimum therapeutic outcome based on patient's requirement. The transdermal formulation or transdermal patch or topical formulation or topical patch of active agents such as nabilone, THC and/or CBD, and derivatives of these compounds can be applied to the skin surface in any of the following dosage regimens such as once in a day, once in two days, once in three days, once in four days, once in five days, once in six days, once in a week, once in a range of from about 8 to about 13 days, once in two weeks, or once in 15 days.

### Example 12

TABLE 1
Formulations of drug in adhesive matrix patch

| Component | % W/W |
|---|---|
| Active component (THC and/or CBD) | 0.5%-30% |
| Solvent | 2%-30% |
| Permeation enhancer | 2%-30% |
| Pressure sensitive adhesive polymer | 20%-80% |
| Polymer | 1%-10% |

### Example 13

TABLE 2
Formulations of drug in adhesive matrix patch

| Component | % W/W |
|---|---|
| Active component (THC and/or CBD) | 1%-30% |
| Solvent | 2%-30% |
| Permeation enhancer | 2%-30% |
| Pressure sensitive adhesive polymer | 20%-80% |

### Example 14

TABLE 3
Formulations of drug in adhesive matrix patch

| Component | % W/W |
|---|---|
| Active component (nabilone) | 0.5%-30% |
| Solvent | 2%-30% |
| Permeation enhancer | 2%-30% |
| Pressure sensitive adhesive polymer | 20%-80% |
| Polymer | 1%-10% |

### Example 15

TABLE 4
Formulations of drug in adhesive matrix patch

| Component | % W/W |
|---|---|
| Active component (nabilone) | 0.5%-30% |
| Solvent | 2%-30% |
| Permeation enhancer | 2%-30% |
| Pressure sensitive adhesive polymer | 20%-80% |

### Example 16

TABLE 5
Formulations of drug in adhesive matrix patch

| Component | % W/W |
|---|---|
| Active component | 1%-30% |
| (THC and/or CBD and/or nabilone) | |
| Solvent | 2%-30% |
| Permeation enhancer | 2%-30% |
| Pressure sensitive adhesive polymer | 20%-80% |

### Example 17

Synthetic delta-9-thc (THC) and cannabidiol (CBD) formulations for transdermal delivery ((Formulation Nos. 001, 002, 003, 004, and 005) were prepared by mixing ingredients as shown in Table 6:

TABLE 6
Transdermal Synthetic Cannabidiol formulations

| | 001 | 002 | 003 | 004 | 005 |
|---|---|---|---|---|---|
| Ingredients | (% W/W) | (% W/W) | (% W/W) | (% W/W) | (% W/W) |
| CBD | 8.0 | 8.0 | 7.7 | 6.9 | 6.4 |
| THC | 8.0 | 8.0 | 7.7 | 6.9 | 6.4 |
| Ethanol | 42.7 | 37.3 | 37.0 | 33.0 | 25.4 |
| Propylene | 40.0 | 40.0 | 37.0 | 33.0 | 30.5 |
| Glycol | | | | | |
| Isopropyl | | | 4.7 | — | — |
| Palmitate | | | | | |
| DMSO | | | | 13.8 | 12.7 |
| Oleic Acid | | 5.3 | 4.7 | 5.5 | 5.1 |
| NMP | | | | — | 12.7 |

Abbreviations:
THCH = delta-9-THC;
CBD = Cannabidiol;
NMP: N-methyl Pyrrolidone.

All of the components from Table 6, with the exception of the CBD and THC, were mixed together with stirring for 18 hours. Next, the CBD and THC were added into the excipient mixture to prepare the final transdermal formulations.

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal CBD and THC formulations prepared as described above. The receptor compartment was filled with receptor medium, held at constant temperature, and constantly stirred to collect the CBD and THC as it diffuses through the skin and into receptor compartment. It is important to confirm that the receptor fluid is always in contact with the skin. The receptor compartment was emptied at 24 hr intervals for assay of CBD and THC and replaced with fresh receptor solution. In order to maintain the sink condition in receptor compartment, it is important to keep the CBD and THC concentration in receptor compartment less than 10% of its solubility. The experimental conditions are provided in Table 7:

TABLE 7
Experimental Condition for In-vitro Permeability testing

| | De-ionized water + 0.5% |
|---|---|
| | Brij-O(20) + 0.01% |
| Receiving Media | Sodium Azide |
| Receiving Media Volume (mL) | 13 |
| Sample Volume (mL) | 13 |
| Sampling Interval (hr) | 24, 48, 72 |
| Franz-cell diffusion area (sqcm) | 1.76 |
| Membrane Type | Human Cadaver Skin |

Flux of CBD and THC through the human cadaver skin was measured for a minimum period of 72 Hrs (3 days) and results of the flux measurement are provided in Table 8 and 9.

TABLE 8
CBD Flux Results

| | 009 | 010 | 011 | 012 | 013 |
|---|---|---|---|---|---|
| Average Flux (0-24 hr) | 0.53 | 0.86 | 1.10 | 1.39 | 1.01 |
| (ug/sqcm/hr) | (23.6%) | (27.0%) | (20%) | (19.9%) | (5.1%) |
| Average Flux (24-48 hr) | 0.92 | 1.16 | 1.21 | 1.44 | 1.21 |
| (ug/sqcm/hr) | (9.6%) | (14.4%) | (18.3%) | (1.65%) | (27.3%) |
| Average Flux (48-72 hr) | 0.52 | 0.86 | 0.71 | 1.27 | 1.01 |
| (ug/sqcm/hr) | (2.44%) | (12.9%) | (13%) | (16.3%) | (27.5%) |
| Average Flux (0-72 hr) | 0.66 | 0.96 | 1.01 | 1.37 | 1.08 |
| (ug/sqcm/hr) | | | | | |

TABLE 9
THC Flux Results

| | 009 | 010 | 011 | 012 | 013 |
|---|---|---|---|---|---|
| Average Flux (0-24 hr) | 0.0 | 0.28 | 0.62 | 0.73 | 0.60 |
| (ug/sqcm/hr) | | (89%) | (20.4%) | (24.5%) | (5.1%) |
| Average Flux (24-48 hr) | 0.39 | 0.65 | 0.78 | 0.85 | 0.80 |
| (ug/sqcm/hr) | (87%) | (20.8%) | (19.4%) | (8.7%) | (32.3%) |
| Average Flux (48-72 hr) | 0.0 | 0.47 | 0.43 | 0.73 | 0.58 |
| (ug/sqcm/hr) | | (20.3%) | (32%) | (20.2%) | (40%) |
| Average Flux (0-72 hr) | 0.13 | 0.47 | 0.61 | 0.77 | 0.66 |
| (ug/sqcm/hr) | | | | | |

### Example 18

Synthetic cannabidiol (CBD) formulations for transdermal delivery ((Formulation Nos. 047-055) were prepared by mixing ingredients as shown in Table 10:

TABLE 10
Transdermal Synthetic Cannabidiol formulations

| Excipients | CBD 047 | CBD 048 | CBD 049 | CBD 050 | CBD 051 | CBD 052 | CBD 053 | CBD 054 | CBD 055 |
|---|---|---|---|---|---|---|---|---|---|
| CBD | 4.84% | 4.98% | 4.73% | 4.99% | 4.87% | 4.89% | 5.04% | 4.83% | 5.00% |
| DURO- | 95.16% | — | — | — | — | — | — | — | — |
| TAK 2516 | | | | | | | | | |
| DURO- | — | 95.02% | — | — | — | — | — | — | — |
| TAK 9301 | | | | | | | | | |
| DURO- | — | — | 95.27% | — | — | — | — | — | — |
| TAK 2287 | | | | | | | | | |
| DURO- | — | — | — | 95.01% | — | — | — | — | — |
| TAK 2054 | | | | | | | | | |
| DURO- | — | — | — | — | 95.13% | — | — | — | — |
| TAK 2852 | | | | | | | | | |
| DURO- | — | — | — | — | — | 95.11% | — | — | — |
| TAK 2074 | | | | | | | | | |
| DURO- | — | — | — | — | — | — | 94.96% | — | — |
| TAK 2194 | | | | | | | | | |
| BIO- | — | — | — | — | — | — | — | 95.17% | — |
| PSA 4501 | | | | | | | | | |
| BIO- | — | — | — | — | — | — | — | — | 95.00% |
| PSA 4201 | | | | | | | | | |

The above ingredients (Table 5) are blended by stirring for 18 hours and then, using a commercial benchtop spreader, the matrix is evenly spread onto an 8×14 inch sheet of release liner (such as 3M 9744) to a thickness of 0.5 mm. The sheet is then place in an oven at 86 F for 120 min to evaporate off the ethyl acetate adhesive solvent. An opaque backing membrane (such as 3M 9730 NR film) with low permeability to oxygen to inhibit photo and oxidative degradation, is then carefully applied by hand to avoid formation of bubbles and voids. A circular die (1.5 inches diameter) is used to cut patches (1.76 sqcm) for subsequent studies. After drying, the drug adhesive matrix has a surface density of 2-30 mg/sqcm, containing CBD in 5% w/w.

The prepared formulations where then subjected to a release study as follows: After weighing the patches (n=3), the release liner was removed, and the patches were placed in 20 ml scintillation vials with 15 ml of receiving media. The receiving media was PBS solution of pH 7.4 with 0.5% Brij(O)20. Vials were placed on the roller overnight at 20 RPM. Samples were withdrawn every 24 hours, up to 72 hours, and media was fully replaced each time. Samples were then run in the HPLC in order to determine the % release of CBD from the different formulations.

The prepared formulations also analyze for the uniformity of drug content. The patches (n=3) were weight out for each formulation, the release liner was removed, and the patches (including the release liner) were placed in 20 ml scintillation vials with 15 ml of solution IPA:Ethanol (190 proof) (50:50). The vials were then placed on the roller at 20 RPM and left overnight. Samples were withdrawn from each vial and analyzed on the HPLC in order to determine the drug content of each formulation.

TABLE 11
Experimental Condition for In-vitro Permeability testing

| | PBS (pH 7.4) + 0.5% |
|---|---|
| | Brij-O(20) + 0.01% |
| Receiving Media | Sodium Azide |
| Receiving Media Volume (mL) | 15 |
| Sample Volume (mL) | 15 |
| Sampling Interval (hr) | 24, 48 |
| Diffusion area (sqcm) | 1.76 |

% Release of CBD through the matrix system was measured for a minimum period of 48Hrs (2 days) and results of the % release are provided in Table 12.

TABLE 12
% CBD Release Results

| | CBD 047 | CBD 048 | CBD 049 | CBD 050 | CBD 051 | CBD 052 | CBD 053 | CBD 054 | CBD 055 |
|---|---|---|---|---|---|---|---|---|---|
| Av. % | 26 (3) | 9 (7) | 20 (7) | 40 (2) | 23 (8) | 35 (12) | 37 (6) | 93 (2) | Did not |
| Release at | | | | | | | | | solubilize |
| 24 hours | | | | | | | | | the CBD |
| Av. % | 18 (7) | 6 (12) | 13 (13) | 22 (1) | 13 (9) | 12 (1) | 22 (5) | 2 (2) | 98% |
| Release at | | | | | | | | | (0.05) |
| 48 hours | | | | | | | | | |
| % Release | 44 | 15 | 33 | 62 | 36 | 47 | 59 | 95 | |
| 0-48 hours | | | | | | | | | |
| (cumulative) | | | | | | | | | |
| % CBD from | 104 (2) | 100 (1) | 100 (5) | 102 (1) | 103 (6) | 96 (15) | 104 (4) | 107 | |
| extraction | | | | | | | | | |
| (STD) | | | | | | | | | |

Drug content study showed that the % recover of CBD in extraction is between 96-107% for all the manufactured formulations. Furthermore, the release study showed that the silicone adhesive 4501 showed more than 90% release within first 24 hrs. Based on the release profile following are the best adhesive for CBD formulation: BIOPSA-4501>2054=2194>2074>2516>2852=2287>9301.
Release studies indicate that the functional group and crosslinker affect the CBD release from acrylic adhesive. According to current study, acrylic adhesive containing —COOH functional group with crosslinker showed the maximum release of CBD from all the acrylic adhesive patches.

### Example 19

Additional synthetic Cannabidiol (CBD) formulations for transdermal delivery (Formulation Nos. 057 through 064) were prepared by mixing ingredients as shown in Table 13:

TABLE 13
Transdermal Synthetic Cannabidiol formulation no. 057 to 064

| Excipients | CBD 057 | CBD 058 | CBD 059 | CBD 060 | CBD 061 | CBD 062 | CBD 063 | CBD 064 |
|---|---|---|---|---|---|---|---|---|
| CBD | 5.0% | 5.0% | 5.0% | 4.9% | 4.8% | 4.8% | 4.9% | 4.8% |
| BIO- | 95.0% | 84.6% | 84.5% | 85.0% | 83.3% | 89.7% | 86.9% | 89.6% |
| PSA 4501 | | | | | | | | |
| IPM | — | 10.4% | — | — | — | — | — | — |
| IPP | — | — | 10.5% | — | — | — | — | — |
| Oleic Acid | — | — | — | 10.1% | — | — | — | — |
| Transcutol P | — | — | — | — | 11.9% | — | — | — |
| Brij O20 | — | — | — | — | — | 5.5% | — | — |
| Poloxamer 124 | — | — | — | — | — | — | 8.2% | — |
| PGML | — | — | — | — | — | — | — | 5.6% |

Synthetic Cannabidiol formulations for transdermal delivery (057-064) were prepared by the same procedure described in Example 17.

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal CBD formulations prepared as described above. The receptor compartment was filled with receptor medium, held at constant temperature, and constantly stirred to collect the CBD as it diffuses through the skin and into receptor compartment. It is important to confirm that the receptor fluid is always in contact with the skin. The receptor compartment was emptied at 24 hr intervals for assay of CBD and replaced with fresh receptor solution. In order to maintain the sink condition in receptor compartment, it is important to keep the CBD concentration in receptor compartment less than 10% of its solubility. The experimental conditions are provided in Table 14:

TABLE 14
Experimental Condition for In-vitro Permeability testing

| | De-ionized water + 0.5% |
|---|---|
| | Brij-O(20) + 0.01% |
| Receiving Media | Sodium Azide |
| Receiving Media Volume (mL) | 13 |
| Sample Volume (mL) | 13 |
| Sampling Interval (hr) | 24, 48, 72, 96 |
| Franz-cell diffusion area (sqcm) | 1.76 |
| Membrane Type | Human Cadaver Skin |

Flux of CBD through the human cadaver skin was measured for a minimum period of 96 Hrs (4 days) and results of the flux measurement are provided in Table 15.
Upon completion of the flux study, the used patches were carefully removed and extract the CBD from the use patches using IPA:Ethanol (50:50). The human cadaver skin was also soaked in IPA:Ethanol (50:50), in order to extract the CBD from it. The samples were analyzed using HPLC. The data in table 6 showed the amount of CBD present in the skin and the left-over patches.

TABLE 15
CBD Flux Results

| | CBD 057 | CBD 058 | CBD 059 | CBD 060 | CBD 061 | CBD 062 | CBD 063 | CBD 064 |
|---|---|---|---|---|---|---|---|---|
| Av. cumulative | 52 | 31 | 16 | 120 | 86 | BLLQ | BLLQ | 53 |
| amount | | | | | | | | |
| permeated | | | | | | | | |
| at 96 hrs (μg) | | | | | | | | |
| Av. flux | 0.41 | 0.37 | 0.37 | 0.69 | 0.51 | BLLQ | BLLQ | 0.42 |
| 24-96 hrs | | | | | | | | |
| (μg/hr/sqcm) | | | | | | | | |
| Peak flux | 0.44 | 0.38 | 0.38 | 0.82 | 0.60 | BLLQ | BLLQ | 0.44 |
| (μg/hr/sqcm) | | | | | | | | |
| Time to peak | 72 | 72 | 72 | 72 | 72 | 0 | 0 | 72 |
| flux (hrs) | | | | | | | | |
| Av. CBD | 1.57 | Did not | | 1.14 | 2.26 | 1.81 | 1.88 | 1.56 |
| Amount in | | Determined | | | | | | |
| patch (mg) | | | | | | | | |
| Av. CBD | 0.01 | | | 0.59 | 0.13 | 0.54 | 0.24 | 0.10 |
| Amount in | | | | | | | | |
| skin (mg) | | | | | | | | |

### Example 20

Additional synthetic Cannabidiol (CBD) formulations for transdermal delivery (Formulation Nos. 102 through 106) were prepared by mixing ingredients as shown in Table 16:

TABLE 16
Transdermal Synthetic Cannabidiol formulation no. 102 to 106

| Excipients | CBD-102 | CBD-103 | CBD-104 | CBD-105 | CBD-106 |
|---|---|---|---|---|---|
| CBD | 3.69% | 4.01% | 3.90% | 3.97% | 3.94% |
| Oleic acid | 3.69% | — | 4.30% | — | 9.06% |
| Propylene | 12.19% | 13.22% | 12.50% | 13.49% | — |
| Glycol | | | | | |
| Isopropyl | — | 4.41% | 4.69% | — | 6.31% |
| Palmitate | | | | | |
| BIO-PSA 4501 | 80.42% | 78.37% | 74.62% | 82.54% | 80.69% |

Synthetic Cannabidiol formulations for transdermal delivery (102-106) were prepared by the same procedure described in Example 17.

The in-vitro flux study was performed by the same procedure described in Example 15. Flux of CBD through the human cadaver skin was measured for a minimum period of 144 Hrs (6 days) and results of the flux measurement are provided in Table 17.

TABLE 17
CBD Flux Results

| Excipients | CBD-102 | CBD-103 | CBD-104 | CBD-105 | CBD-106 |
|---|---|---|---|---|---|
| Avg Flux 0-24 hr, | 0.23 (0.37) | 0.51 (87.37) | 0.12 (173.21) | 0.13 (173.21) | 0.00 |
| μg/sqcm/hr (% RSD) | | | | | |
| Avg Flux 24-90 hr, | 0.44 (8.00) | 0.30 (2.80) | 0.32 (14.52) | 0.31 (12.19) | 0.30 (22.80) |
| μg/sqcm/hr (% RSD) | | | | | |
| Avg Flux 90-120 hr, | 0.53 (10.96) | 0.40 (13.95) | 0.41 (13.50) | 0.35 (13.20) | 0.36 (16.54) |
| μg/sqcm/hr (% RSD) | | | | | |
| Avg Flux 120-144 hr, | 0.51 (9.02) | 0.42 (11.41) | 0.44 (8.42) | 0.27 (15.04) | 0.27 (87.40) |
| μg/sqcm/hr (% RSD) | | | | | |
| Avg Flux 24-144 hr, | 0.43 (7.09) | 0.41 (28.88) | 0.32 (12.14) | 0.26 (13.47) | 0.23 (31.69) |
| μg/sqcm/hr (% RSD) | | | | | |

### Example 21

Additional synthetic Cannabidiol (CBD) formulations for transdermal delivery (Formulation No. 060) were prepared by mixing ingredients as shown in Table 13:

TABLE 18
Transdermal Synthetic Cannabidiol
formulation no. 60

| Excipients | CBD-060 |
|---|---|
| CBD | 4.84% |
| Oleic acid | 10.01% |
| BIO-PSA 4501 | 85.15% |

Synthetic Cannabidiol formulations for transdermal delivery (060) were prepared by the same procedure described in Example 14.

The in-vitro flux study was performed by the same procedure described in Example 18. The effect of receiving media (RM) was evaluated using several different RM: 1) 0.5% w/w Brij 020 in water, 2) 1% w/w Brij O20 in water, 3) 0.5% w/w Brij O20 in water:Ethanol (50:50), 4) 0.5% w/w Brij O20 in Water:Ethanol (90:10), 5) 0.5% w/w Brij O20 in Water:Ethanol (80:20), 6) 0.5% w/w Brij O20 in Water:Ethanol (70:30) and, 7) 0.5% w/w Brij O20 in Water:PEG400 (60:40). Flux of CBD through the human cadaver skin was measured for a minimum period of 168 Hrs (7 days) and results of the flux measurement are provided in Table 19, Table 20 and Table 21.

TABLE 19
CBD Flux Results

| | | | DI water with |
|---|---|---|---|
| | DI water + | DI water + | 0.5% Brij O20 |
| | 0.5% Brij | 1.0% Brij | (50%) + ethanol |
| Receiving Media | O20 | O20 | (50%) |
| Avg Flux 0-24 hr, | BLLQ | BLLQ | 6.81 (12.65) |
| μg/sqcm/hr (% RSD) | | | |
| Avg Flux 24-48 hr, | BLLQ | BLLQ | 5.01 (10.46) |
| μg/sqcm/hr (% RSD) | | | |
| Avg Flux 48-72 hr, | BLLQ | BLLQ | 3.73 (21.04) |
| μg/sqcm/hr (% RSD) | | | |
| Avg Flux 72-96 hr, | BLLQ | BLLQ | 1.24 (21.31) |
| μg/sqcm/hr (% RSD) | | | |
| Avg Flux 96-120 hr, | BLLQ | BLLQ | 4.14 (22.31) |
| μg/sqcm/hr (% RSD) | | | |
| Avg Flux 120-144 hr, | BLLQ | BLLQ | 3.08 (31.94) |
| μg/sqcm/hr (% RSD) | | | |
| Avg Flux 144-168 hr, | BLLQ | BLLQ | 0.55 (81.13) |
| μg/sqcm/hr (% RSD) | | | |

TABLE 20
CBD Flux Results

| | DI water with | DI water with | DI water with |
|---|---|---|---|
| | 0.5% Brij O20 | 0.5% Brij O20 | 0.5% Brij O20 |
| | (90%) + ethanol | (80%) + ethanol | (50%) + ethanol |
| Receiving Media | (10%) | (20%) | (50%) |
| Avg Flux 0-24 hr, | BLLQ | 0.15 (154.97) | 5.58 (24.33) |
| μg/sqcm/hr (% RSD) | | | |
| Avg Flux 24-48 hr, | 0.24 (111.0) | 0.13 (154.92) | 4.20 (11.02) |
| μg/sqcm/hr (% RSD) | | | |
| Avg Flux 48-72 hr, | 0.15 (155.34) | 0.08 (244.95) | 5.16 (25.90) |
| μg/sqcm/hr (% RSD) | | | |
| Avg Flux 72-96 hr, | 0.27 (77.80) | 0.08 (244.95) | 4.01 (18.19) |
| μg/sqcm/hr (% RSD) | | | |
| Avg Flux 96-120 hr, | 0.49 (10.47) | 0.15 (155.68) | 3.37 (13.85) |
| μg/sqcm/hr (% RSD) | | | |
| Avg Flux 120-144 hr, | 0.42 (49.57) | 0.16 (155.47) | 2.54 (29.27) |
| μg/sqcm/hr (% RSD) | | | |
| Avg Flux 144-168 hr, | BLLQ | 0.18 (138.85) | 0.44 (99.0) |
| μg/sqcm/hr (% RSD) | | | |

TABLE 21
CBD Flux Results

| | DI water with | DI water with | DI water |
|---|---|---|---|
| | 0.5% Brij O20 | 0.5% Brij O20 | (60%) + |
| | (70%) + ethanol | (50%) + ethanol | PEG-400 |
| Receiving Media | (30%) | (50%) | (40%) |
| Avg Flux 0-24 hr, | 0.36 (79.23) | 4.49 (37.81) | BLLQ |
| μg/sqcm/hr (% RSD) | | | |
| Avg Flux 24-48 hr, | 1.23 (31.26) | 11.69 (8.97) | 0.49 (18.12) |
| μg/sqcm/hr (% RSD) | | | |
| Avg Flux 48-72 hr, | 1.68 (27.58) | 13.07 (84.62) | 0.62 (14.70) |
| μg/sqcm/hr (% RSD) | | | |
| Avg Flux 72-96 hr, | 1.32 (22.94) | 5.80 (33.13) | 0.62 (13.38) |
| μg/sqcm/hr (% RSD) | | | |
| Avg Flux 96-120 hr, | 0.98 (20.19) | 3.19 (42.69) | 0.66 (14.62) |
| μg/sqcm/hr (% RSD) | | | |

### Example 22

Additional synthetic Cannabidiol (CBD) formulations for transdermal delivery (Formulation No. 102 and 104) were prepared by mixing ingredients as shown in Table 22:

TABLE 22
Transdermal Synthetic Cannabidiol
formulation no. 102 and 104

| Excipients | CBD-102 | CBD-104 |
|---|---|---|
| CBD | 4.34 | 4.21 |
| PG | 13.45 | 13.04 |
| Oleic Acid | 4.34 | 4.63 |
| IPP | — | 4.21 |
| BIO-PSA 4501 | 77.86 | 73.91 |

Synthetic Cannabidiol formulations for transdermal delivery (102 and 104) were prepared by the same procedure described in Example 17.

The in-vitro flux study was performed by the same procedure described in Example 2. The effect of receiving media (RM) was evaluated using two different RM: 1) 0.5% w/w Brij O20 in water, and 2) 0.5% w/w Brij O20 in water:Ethanol (50:50. Flux of CBD through the human cadaver skin was measured for a minimum period of 120-144 Hrs (5-6 days) and results of the flux measurement are provided in Table 23.

TABLE 23
CBD Flux Results

| | Formulation | | | |
|---|---|---|---|---|
| | CBD-102 | | CBD-104 | |
| | | DI water with | | DI water with |
| | | 0.5% Brij O20 | | 0.5% Brij O20 |
| | DI water + | (50%) + ethanol | DI water + | (50%) + ethanol |
| Receiving Media | 0.5% Brij O20 | (50%) | 0.5% Brij O20 | (50%) |
| Avg Flux 0-24 hr, | BLLQ | 4.10 (29.62) | BLLQ | 8.54 (9.22) |
| μg/sqcm/hr (% RSD) | | | | |
| Avg Flux 24-48 hr | BLLQ | 9.94 (1.99) | BLLQ | 9.67 (12.07) |
| μg/sqcm/hr (% RSD) | | | | |
| Avg Flux 48-72 hr, | 0.52 (10.97) | 13.87 (1.61) | BLLQ | 5.62 (24.78) |
| μg/sqcm/hr (% RSD) | | | | |
| Avg Flux 72-96 hr, | 0.42 (10.54) | 2.80 (4.14) | BLLQ | 3.66 (40.98) |
| μg/sqcm/hr (% RSD) | | | | |
| Avg Flux 96-120 hr, | 0.40 (7.27) | 16.27 (13.12) | BLLQ | 4.66 (45.26) |
| μg/sqcm/hr (% RSD) | | | | |
| Avg Flux 120-144 hr, | — | — | BLLQ | 2.20 (61.51) |
| μg/sqcm/hr (% RSD) | | | | |

While the invention has been described in detail and with reference to specific examples thereof, it will be apparent to one skilled in the art that various changes and modifications can be made therein without departing from the spirit and scope thereof.

1. A pharmaceutical composition comprising an active agent selected from the group consisting of tetrahydrocannabinol (THC), cannabidiol (CBD), nabilone, the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorphs thereof, ion-pairs thereof, stereoisomers thereof, racemic forms thereof, and combinations thereof, in a dosage form for transdermal delivery.

2. The pharmaceutical composition of claim 1 which comprises at least about 0.1% to about 80% (w/w) of active agent.

3. The pharmaceutical composition of claim 1 which comprises at least about 0.5-30% of active agent.

4. The pharmaceutical composition of claim 1 which comprises active agent at a concentration of, independently, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80%.

5. The pharmaceutical composition of claim 1 formulated as transdermal liquid formulation, transdermal semisolid formulation, transdermal gel formulation, or transdermal polymer matrix formulation, transdermal adhesive matrix formulation, film forming gel, film forming spray formulation, transdermal drug in adhesive matrix formulation.

6. The pharmaceutical composition of claim 1 further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, diluents, plasticizers, surfactants, antioxidants, oxidants, and combinations thereof.

7. The pharmaceutical composition of claim 1 further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, diluents, plasticizers, surfactants, antioxidants, oxidants, and combinations thereof in the range of 0.1%-99.5% w/w or w/v.

8. The pharmaceutical composition of claim 1 wherein the carrier is present in the range of 5%-99% w/w or w/v.

9. The pharmaceutical composition of claim 1 which is formulated as a transdermal patch.

10. The pharmaceutical composition of claim 1 formulated as a transdermal patch, wherein the transdermal patch is selected from the group such as to reservoir patch, a microreservoir patch, a matrix patch, a pressure sensitive adhesive patch, extended release transdermal film a liquid reservoir system, a microreservoir patch, a matrix patch, a pressure sensitive adhesive patch, a mucoadhesive patch, transdermal film forming formulation.

11. The pharmaceutical composition of claim 1 indicated for the treatment and/or prevention and/or control of chronic pain in a patient.

12. The pharmaceutical composition of claim 1 which is formulated as the transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, once in 15 days to about 30 days.

13. The pharmaceutical composition of claim 1 which may be formulated as microneedles.

14. The pharmaceutical composition of claim 1 wherein said of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof is produced by a synthetic route.

15. A method for the treatment and/or prevention and/or control of chronic pain in a patient comprising:
selecting a patient in need of treatment and/or prevention and/or control of chronic pain;
topically applying the topical pharmaceutical composition of claim 1.

16. The method of claim 15 wherein the chronic pain is selected from the group consisting of neuropathic pain, peripheral neuropathic pain, inflammatory pain, musculoskeletal pain, pain due to muscle spasms, pain due to increased muscle tone, osteoarthritic pain, muscular headache, tension-type headache, migraine, cluster headache, atypical facial pain, referred pain, vulvodynia, proctodynia, and any combination thereof.

17. The method of claim 15 wherein the topical application of a transdermal pharmaceutical composition is for the treatment and/or prevention and/or control of chronic pain in a patient, and wherein the transdermal patch is applied at a time period selected from the group consisting of once in a day, once in two days, once in three days, once in four days, once in five days, once in six days, once in a week, and once in ten days, and once in fifteen days, and wherein transdermal film formulation is applied at a time period selected from the group consisting of once in a day, once in 10-20 hours, once in 5-10 hours, once in 5 hours.

18. The method of claim 15 further providing a constant rate of delivery of the active components of the transdermal patch, transdermal film forming formulation over a time period.

19. The method of claim 15 further providing a steady absorption rates of the active components of the transdermal patch, transdermal film forming formulation over a time period.

20. The method of claim 15 further achieving a constant blood serum levels of the active components of the transdermal patch, transdermal film forming formulation over a time period.

21. The method of claim 15 further achieving a reduced variability in dosage of the active components of the transdermal patches, transdermal film forming formulation over a time period.

22. The method of claim 15 further providing a plasma concentration of the active components of the transdermal patch, transdermal film forming formulation in a therapeutic range.

23. The method of claim 15 further providing a plasma concentration of the active components of the transdermal patch, transdermal film forming formulation in a range about 0.01 ng/mL to about 500 ng/mL.

24. The method of claim 15 further providing a plasma concentration of the active components of the transdermal patch, transdermal film forming formulation in a range of about 0.1 ng/mL to about 300 ng/mL.

25. A method for the treatment and/or prevention and/or control of multiple sclerosis, multiple sclerosis-related muscle spasms, and pain and/or spasticity in multiple sclerosis in a patient comprising:
selecting a patient in need of treatment and/or prevention and/or control of multiple sclerosis, multiple sclerosis-related muscle spasms, pain and/or spasticity in multiple sclerosis; —topically applying the transdermal pharmaceutical composition of claim 1.

26. The method of claim 25 wherein the topical application of a transdermal pharmaceutical composition for the treatment and/or prevention and/or control of multiple sclerosis, multiple sclerosis-related muscle spasms, pain and/or spasticity in multiple sclerosis in a patient, wherein the transdermal patch is applied at a time period selected from the group consisting of once in a day, once in two days, once in three days, once in four days, once in five days, once in six days, once in a week, and once in ten days, and wherein transdermal film formulation is applied at a time period selected from the group consisting of once in a day, once in 10-20 hours, once in 5-10 hours, once in 5 hours.

27. The method of claim 25 further providing a constant rate of delivery of the active components of the transdermal patch, transdermal film formulation over a time period.

28. The method of claim 25 further providing a steady absorption rates of the active components of the transdermal patch, transdermal film formulation over a time period.

29. The method of claim 25 further achieving a constant blood serum levels of the active components of the transdermal patch, transdermal film formulation over a time period.

30. The method of claim 25 further achieving a reduced variability in dosage of the active components of the transdermal patches, transdermal film formulation over a time period.

31. The method of claim 25 further providing a plasma concentration of the active components of the transdermal patch, transdermal film formulation in a therapeutic range over a period of time.

32. The method of claim 25 further providing a plasma concentration of the active components of the transdermal patch, transdermal film formulation in a therapeutic range of about 0.01 ng/mL to about 500 ng/mL.

33. The method of claim 25 further providing a plasma concentration of the active components of the transdermal patch, transdermal film forming formulation in a range of about 0.1 ng/mL to about 300 ng/mL.

34. A method for the treatment and/or prevention and/or control of chemotherapy-induced nausea and vomiting (CINV) in a patient comprising:
selecting a patient in need of treatment and/or prevention and/or control of CINV;
topically applying the transdermal pharmaceutical composition of claim 1.

35. The method of claim 34, wherein the CINV is selected from the group consisting of acute CINV, delayed CINV, chronic CINV, Breakthrough CNV, refractory CINV, and any combination thereof.

36. The method of claim 34 wherein the topical application of a transdermal pharmaceutical composition is for the treatment and/or prevention and/or control of CINV in a patient, and wherein the transdermal patch is applied at a time period selected from the group consisting of once in a day, once in two days, once in three days, once in four days, once in five days, once in six days, once in a week, and once in ten days. and wherein transdermal film formulation is applied at a time period selected from the group consisting of once in a day, once in 10-20 hours, once in 5-10 hours, once in 5 hours.

37. The method of claim 34 further providing a constant rate of delivery of the active components of the transdermal patch, transdermal film formulation over a time period.

38. The method of claim 34 further providing a steady absorption rates of the active components of the transdermal patch, transdermal film formulation over a time period.

39. The method of claim 34 further achieving a constant blood serum levels of the active components of the transdermal patch, transdermal film formulation over a time period.

40. The method of claim 34 further achieving a reduced variability in dosage of the active components of the transdermal patches, transdermal film formulation over a time period.

41. The method of claim 34 further providing a plasma concentration of the active components of the transdermal patch, transdermal film formulation in a therapeutic range over a period of time.

42. The method of claim 34 further providing a plasma concentration of the active components of the transdermal patch, transdermal film forming formulation in a range of about 0.01 ng/mL to about 500 ng/mL.

43. A method for the treatment and/or prevention and/or control of epilepsy in a patient comprising:
selecting a patient in need of treatment and/or prevention and/or control of epilepsy;
topically applying the transdermal pharmaceutical composition of claim 1.

44. The method of claim 43, wherein the epilepsy is selected from the group consisting of juvenile myoclonic epilepsy, Lennox-Gastaut, Dravet syndrome, mesial temporal lobe epilepsy, nocturnal frontal lobe epilepsy, progressive epilepsy with mental retardation, progressive myoclonic epilepsy, as well as seizures associated with CNS mass lesions, and any combination thereof.

45. The method of claim 43 wherein the topical application of a transdermal pharmaceutical composition is for the treatment and/or prevention and/or control of epilepsy in a patient, and wherein the transdermal patch is applied at a time period selected from the group consisting of once in a day, once in two days, once in three days, once in four days, once in five days, once in six days, once in a week, and once in ten days, and wherein transdermal film formulation is applied at a time period selected from the group consisting of once in a day, once in 10-20 hours, once in 5-10 hours, once in 5 hours.

46. The method of claim 43 further providing a constant rate of delivery of the active components of the transdermal patch, transdermal film formulation over a time period.

47. The method of claim 43 further providing a steady absorption rates of the active components of the transdermal patch, transdermal film formulation over a time period.

48. The method of claim 43 further achieving a constant blood serum levels of the active components of the transdermal patch, transdermal film formulation over a time period.

49. The method of claim 43 further achieving a reduced variability in dosage of the active components of the transdermal patches, transdermal film formulation over a time period.

50. The method of claim 43 further providing a plasma concentration of the active components of the transdermal patch, transdermal film forming formulation in a therapeutic range over a period of time.

51. The method of claim 43 further providing a plasma concentration of the active components of the transdermal patch, transdermal film forming formulation, in a range of about 0.01 ng/mL to about 500 ng/mL.

52. A method for the treatment and/or prevention and/or control of seizure disorder in a patient comprising:
selecting a patient in need of treatment and/or prevention and/or control of seizure disorder;
topically applying the transdermal pharmaceutical composition of claim 1.

53. The method of claim 52, wherein the seizure disorder is selected from the group consisting of complex partial seizures, simple partial seizures, partial seizures with secondary generalization, generalized seizures (including absence, grand mal (tonic clonic), status epilepticus, tonic, atonic, myoclonic), neonatal and infantile spasms, drug-induced seizures, trauma-induced seizures, febrile seizures, and any combination thereof.

54. The method of claim 52 wherein the topical application of a transdermal pharmaceutical composition is for the treatment and/or prevention and/or control of seizure disorder in a patient, and wherein the transdermal patch is applied at a time period selected from the group consisting of once in a day, once in two days, once in three days, once in four days, once in five days, once in six days, once in a week, and once in ten days, and wherein transdermal film formulation is applied at a time period selected from the group consisting of once in a day, once in 10-20 hours, once in 5-10 hours, once in 5 hours.

55. The method of claim 52 further providing a constant rate of delivery of the active components of the transdermal patch, transdermal film formulation over a time period.

56. The method of claim 52 further providing a steady absorption rates of the active components of the transdermal patch, transdermal film formulation over a time period.

57. The method of claim 52 further achieving a constant blood serum levels of the active components of the transdermal patch, transdermal film formulation over a time period.

58. The method of claim 52 further achieving a reduced variability in dosage of the active components of the transdermal patches, transdermal film formulation over a time period.

59. The method of claim 52 further providing a plasma concentration of the active components of the transdermal patch, transdermal film forming formulation, in a therapeutic range over a period of time.

60. The method of claim 52 further providing a plasma concentration of the active components of the transdermal patch, transdermal film formulation in a range of about 0.01 ng/mL to about 500 ng/mL.

61. A method for the treatment and/or prevention and/or control of neurological disorder in a patient comprising:
selecting a patient in need of treatment and/or prevention and/or control of neurological disorder;
topically applying the transdermal pharmaceutical composition of claim 1.

62. The method of claim 61, wherein the neurological disorder is selected from the group consisting of cerebral ischemia, ischemia, stroke, neurodegeneration, neurological complications arising from such as Alzheimer's disease, Parkinson's disease, Wilson's disease, Lewy body dementia, multiple sclerosis, seizure disorders, cerebellar ataxia, progressive supranuclear palsy, amyotrophic lateral sclerosis, autism, affective disorders, anxiety disorders, metabolic disorders that affect the CNS, and/or schizophrenia; cell damage; nerve damage from cerebrovascular disorders such as stroke in the brain or spinal cord, from CNS infections including meningitis and HIV, from tumors of the brain and spinal cord, prion diseases, and CNS disorders resulting from ordinary aging (e.g., anosmia), head and/or brain injury, or spinal cord injury, and any combination thereof.

63. The method of claim 61 wherein the topical application of a transdermal pharmaceutical composition is for the treatment and/or prevention and/or control of neurological disorder in a patient, and wherein the transdermal patch is applied at a time period selected from the group consisting of once in a day, once in two days, once in three days, once in four days, once in five days, once in six days, once in a week, and once in ten days, and wherein transdermal film formulation is applied at a time period selected from the group consisting of once in a day, once in 10-20 hours, once in 5-10 hours, once in 5 hours.

64. The method of claim 61 further providing a constant rate of delivery of the active components of the transdermal patch, transdermal film forming formulation over a time period.

65. The method of claim 61 further providing a steady absorption rates of the active components of the transdermal patch over a time period.

66. The method of claim 61 further achieving a constant blood serum levels of the active components of the transdermal patch, transdermal film forming formulation, over a time period.

67. The method of claim 61 further achieving a reduced variability in dosage of the active components of the transdermal patches, transdermal film forming formulation over a time period.

68. The method of claim 61 further providing a plasma concentration of the active components of the transdermal patch, transdermal film forming formulation in a therapeutic range over a period of time.

69. The method of claim 61 further providing a plasma concentration of the active components of the transdermal patch, transdermal film forming formulation in a range of about 0.01 15 ng/mL to about 500 ng/mL.
